(19) United States
(12) Patent Application Publication
(10) Pub. No.: US 2009/0286772 A1
(43) Pub. Date: Nov. 19, 2009

(54) 2-(Phenyl or Heterocyclic)-1H-Phenanthro[9,10-D]Imidazoles

(76) Inventors: Anh Chau, Saint-Laurent (CA); Bernard Cote, L'Ile-Perrot (CA); Yves Ducharme, Montreal (CA); Richard Frenette, Laval (CA); Richard Friesen, Kirkland (CA); Marc Gagnon, Montreal (CA); Andre Giroux, Ste-Anne-De-Bellevue (CA); Evelyn Martins, Vaudreuil (CA); Hongping Yu, Kirkland (CA); Pierre Hamel, Vimont-Laval (CA)

Correspondence Address: MERCK AND CO., INC, P O BOX 2000, RAHWAY, NJ, 07065-0907, US

(21) Appl. No.: 12/224,275

(22) PCT Filed: Feb. 22, 2007
(86) PCT No.: PCT/CA2007/000287

§ 371 Date: Aug. 22, 2008

Related U.S. Application Data
(60) Provisional application No. 60/776,392, filed on Feb. 24, 2006.
(60) Provisional application No. 60/796,803, filed on May 2, 2006.

(51) Int. Cl. A61K 31/4184; C07D 235/04; C07D 401/04; C07D 413/02; C07D 417/02; A61K 31/4439; A61K 31/5377; A61K 31/541; A61P 25/00; A61P 19/00

(52) U.S. Cl. 514/228.2; 548/301.7; 546/273.1; 544/139; 544/60; 514/394; 514/338; 514/232.8

(57) ABSTRACT

The invention encompasses novel compounds of Formula (I) or pharmaceutically acceptable salts thereof. These compounds are inhibitors of the microsomal prostaglandin E synthase-1 (mPGES-1) enzyme and are therefore useful to treat pain and/or inflammation from a variety of diseases or conditions, such as osteoarthritis, rheumatoid arthritis and acute or chronic pain. Methods of treating diseases or conditions mediated by the mPGES-1 enzyme and pharmaceutical compositions are also encompassed.

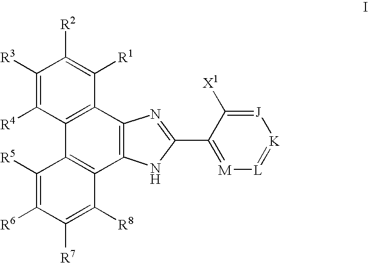

### BACKGROUND OF THE INVENTION

Modulation of prostaglandin metabolism is at the center of current anti-inflammatory therapies. NSAIDs and COX-2 inhibitors block the activity of cyclooxygenases and their ability to convert arachidonic acid (AA) into prostaglandin (PG) H2. PGH2 can be subsequently metabolized by terminal prostaglandin synthases to the corresponding biologically active PGs, namely, PGI2, thromboxane (Tx) A2, PGD2, PGF2α, and PGE2. A combination of pharmacological, genetic, and neutralizing antibody approaches demonstrates the importance of PGE2 in inflammation. In many respects, disruption of PGE2-dependent signalling in animal models of inflammation can be as effective as treatment with NSAIDs or COX-2 inhibitors. The conversion of PGH2 to PGE2 by prostaglandin E synthases, (PGES) may therefore represent a pivotal step in the propagation of inflammatory stimuli.

Microsomal prostaglandin E synthase-1 (mPGES-1) is an inducible PGES after exposure to pro-inflammatory stimuli. mPGES-1 is induced in the periphery and in the CNS by inflammation and represents therefore a novel target for acute and chronic inflammatory disorders. The rationale for the development of specific mPGES-1 inhibitors revolves around the hypothesis that the therapeutic utility of NSAIDs and Cox-2 inhibitors would be largely due to inhibition of pro-inflammatory PGE2 while the side effect profile would be largely due to inhibition of other prostaglandins.

The present invention is directed to novel compounds that are selective inhibitors of the microsomal prostaglandin E synthase-1 enzyme and would therefore be useful for the treatment of pain and inflammation in a variety of diseases or conditions, such as osteoarthritis, rheumatoid arthritis and acute or chronic pain. Furthermore, by selectively inhibiting the pro-inflammatory PGE2, it is believed the compounds of the invention would have a reduced potential for side effects associated with the inhibition of other prostaglandins by conventional non-steroidal anti-inflammatory drugs, such as gastrointestinal and renal toxicity.

### SUMMARY OF THE INVENTION

The invention encompasses novel compounds of Formula I

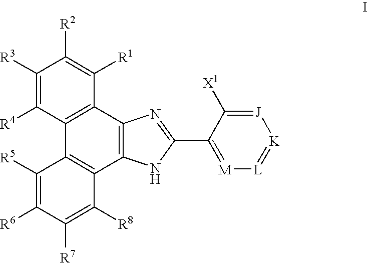

or pharmaceutically acceptable salts thereof. These compounds are inhibitors of the microsomal prostaglandin E synthase-1 (mPGES-1) enzyme and are therefore useful to treat pain and/or inflammation from a variety of diseases or conditions, such as osteoarthritis, rheumatoid arthritis and acute or chronic pain. Methods of treating diseases or conditions mediated by the mPGES-1 enzyme and pharmaceutical compositions are also encompassed.

### DETAILED DESCRIPTION OF THE INVENTION

The invention encompasses a genus of compounds represented by Formula I

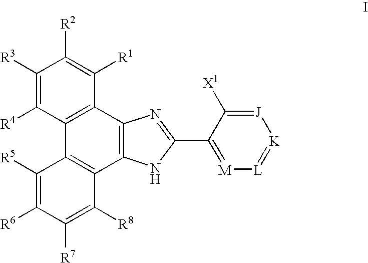

or a prodrug thereof, or a pharmaceutically acceptable salt of said compound or prodrug, wherein:
J is selected from the group consisting of —C(X²)— and —N—,
K is selected from the group consisting of —C(X³)— and —N—,
L is selected from the group consisting of —C(X⁴)— and —N—, and
M is selected from the group consisting of —C(X⁵)— and —N—,
with the proviso that at least one of J, K, L or M is other than —N—, and with the proviso that when J is —C(X²)—, K is —C(X³)—, L is —C(X⁴)—, M is —C(X⁵)— and X⁵ is H, then at least one of R³ and R⁶ is other than H;
X¹ is selected from the group consisting of: (1) F; (2) Cl; (3) Br; (4) I; (5) —N₃; (6) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy group; (7) C₁₋₄alkoxy; (8) NR⁹R¹⁰—C(O)—C₁₋₄-alkyl-O—; (9) C₁₋₄alkyl-S(O)ₖ—; (10) —NO₂; (11) C₃₋₆cycloalkyl, (12) C₃₋₆cycloalkoxy; (13) phenyl, (14) carboxy; and (15) C₁₋₄alkyl-O—C(O)—;
X², X³, X⁴ and X⁵ are independently selected from the group consisting of: (1) H; (2) F; (3) Cl; (4) Br; (5) I; (6) —OH; (7) —N₃; (8) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (9) C₁₋₄alkoxy; (10) NR⁹R¹⁰—, NR⁹R¹⁰—C(O)—C₁₋₄alkyl-O— or NR⁹R¹⁰—C(O)—; (11) C₁₋₄alkyl-S(O)ₖ—; (12) —NO₂; (13) C₃₋₆cycloalkyl, (14) C₃₋₆cycloalkoxy; (15) phenyl, (16) carboxy; and (17) C₁₋₄alkyl-O—C(O)—;
R¹, R², R³, R⁴, R⁵, R⁶, R⁷ and R⁸ are independently selected from the group consisting of: (1) H; (2) F; (3) Cl; (4) Br; (5) I; (6) —CN; (7) C₁₋₆alkyl or C₂₋₆alkenyl, wherein one or more of the hydrogen atoms attached to said C₁₋₆alkyl or C₂₋₆alkenyl may be replaced with a fluoro atom, and wherein said C₁₋₆alkyl or C₂₋₆alkenyl may be optionally substituted with one to three substituents independently selected from the group consisting of: —OH, methoxy, R¹¹—O—C(O)—, cyclopropyl, pyridyl and phenyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C; (18) —(CH₃)C═N—OH or —(CH₃)C═N—OCH₃; and (19) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with 1 to 3 substituents independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₄alkyl and phenyl optionally substituted with 1 to 3 groups independently selected from halo and hydroxy; or R⁵ and R⁶ or R⁷ and R⁸ may be joined together with the carbon atoms to which they are attached to form phenyl;
each Z is independently selected from the group consisting of: (1) H; (2) C₁₋₆alkyl, wherein one or more of the hydrogen atoms attached to said C₁₋₆alkyl may be replaced with a fluoro atom, and wherein C₁₋₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or —(CH₃)C═N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and (2) C₁₋₄alkyl;
each R¹¹, R¹², R¹³, R¹⁴, R¹⁶, R²³, R²⁵, R³⁰ and R³¹ is independently selected from the group consisting of: (1) H; (2) C₁₋₄alkyl, (3) C₃₋₆cycloalkyl; (4) phenyl, (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br, I and methyl;
each R¹⁷, R¹⁸, R¹⁹, R²⁰, R²¹, R²², R²⁶, R²⁷, R²⁸ and R²⁹ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹ may be joined together with the nitrogen atom to which they are attached to form a monocyclic ring of 5 or 6 carbon atoms, optionally containing one or two atoms independently selected from —O—, —S(O)ₖ— and —N(R³²)—; and
each k is independently 0, 1 or 2.

Within this genus, the invention encompasses a sub-genus of compounds represented by Formula A

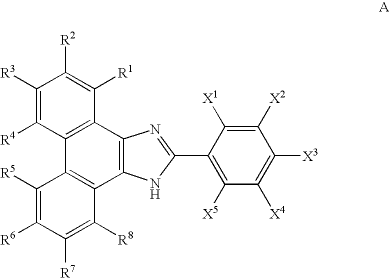

or a producing thereof, or a pharmaceutically acceptable salt of said compound or prodrug.

Within this sub-genus, the invention encompasses a class of compounds of Formula A wherein: X¹ is selected from the group consisting of: (1) F; (2) Cl; (3) Br; and (4) I; and X², X³, X⁴ and X⁵ are independently selected from the group consisting of: (1) H; (2) F; (3) Cl; (4) Br; and (5) I.

Also within this sub-genus, the invention encompasses a class of compounds of Formula A wherein X², X³ and X⁴ are H, and X⁵ is other than H. Within this class, the invention encompasses a sub-class of compounds of Formula A wherein X¹ and X⁵ are the same and selected from the group consisting of: (1) F; (2) Cl; (3) Br; and (4) I.

Also within this sub-genus, the invention encompasses a class of compounds of Formula A wherein at least one of R¹ or R⁸ is other than H.

Also within this sub-genus, the invention encompasses a class of compounds of Formula A wherein at least one of R² or R⁷ is other than H.

Also within this sub-genus, the invention encompasses a class of compounds of Formula A wherein at least one of R⁴ or R⁵ is other than H.

Also within this sub-genus, the invention encompasses a class of compounds of Formula A wherein: at least one of R³ or R⁶ is other than H; and R¹, R², R⁴, R⁵, R⁷ and R⁸ are H. Within this class, the invention encompasses a sub-class of compounds of Formula A wherein R³ and R⁶ are both other than H. Within this sub-class, the invention encompasses compounds of Formula A wherein: one of R³ or R⁶ is independently selected from the group consisting of: F, Cl, Br and I; and the other of R³ or R⁶ is Z-C≡C. Also within this class, the invention encompasses a sub-class of compounds of Formula A wherein: R³ and R⁶ are independently selected from the group consisting of: hydrogen, fluoro, chloro, bromo, iodo, cyano, methyl, ethyl, vinyl, cyclopropyl, —CO₂i-Pr, —CO₂CH₃, —SO₂CF₃, 3-pyridyl, acetyl,

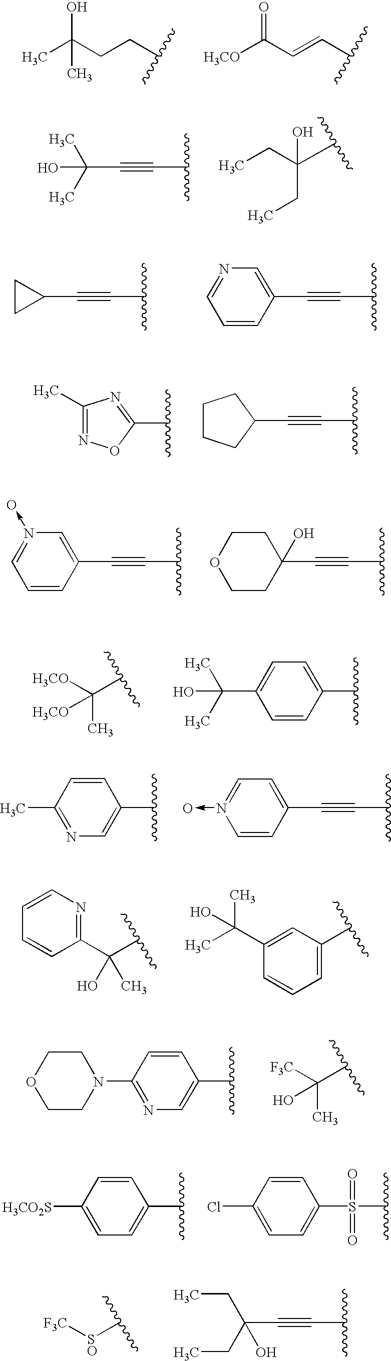

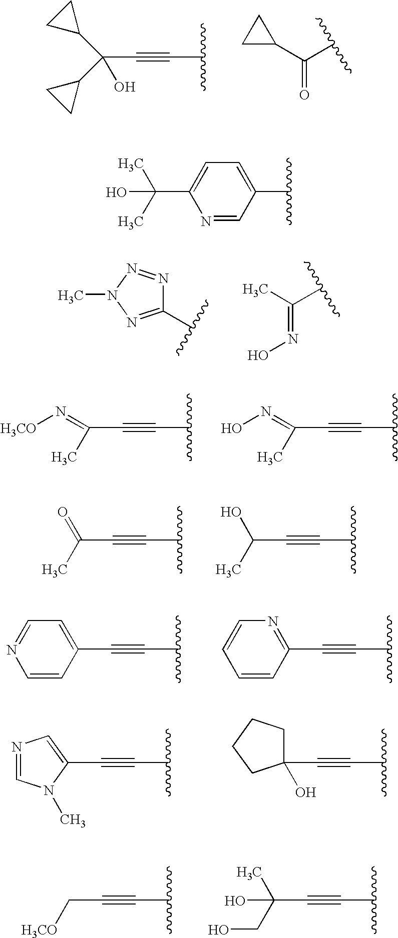

with the proviso that at least one of R³ or R⁶ is other than H.

Within the genus previously described, the invention encompasses a sub-genus of compounds of Formula B:

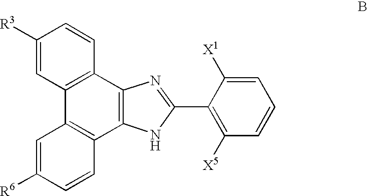

or a prodrug thereof, or a pharmaceutically acceptable salt of said compound or prodrug, wherein: X¹ and X⁵ are independently selected from the group consisting of: (1) F; (2) Cl; (3) Br; and (4) I. Within this sub-genus, the invention encompasses a class of compounds of Formula B wherein: one of R³ or R⁶ is independently selected from the group consisting of: F, Cl, Br and I; and the other of R³ or R⁶ is Z-C≡C.

Within the genus previously described, the invention encompasses a sub-genus of compounds of Formula I in accordance with Formula C

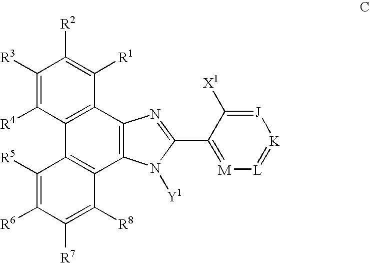

or a pharmaceutically acceptable salt thereof, wherein:
Y¹ is selected from the group consisting of: (1) C₁₋₆alkyl; (2) PO₄—C₁₋₄alkyl-; (3) C₁₋₄alkyl-C(O)—O—CH₂—, wherein the C₁₋₄alkyl portion is optionally substituted with R³³—O—C(O)—; and (4) C₁₋₄alkyl-O—C(O)—; and
R³³ is selected from the group consisting of: (1) H; (2) C₁₋₄alkyl, (3) C₃₋₆cycloalkyl; (4) phenyl; (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br and I.
The invention also encompasses a pharmaceutical composition comprising a compound of Formula I in combination with a pharmaceutically acceptable carrier.

The invention also encompasses a method for treating a microsomal prostaglandin E synthase-1 mediated disease or condition in a human patient in need of such treatment comprising administering to said patient a compound according to claim 1 in an amount effective to treat the microsomal prostaglandin E synthase-1 mediated disease or condition. Within this embodiment is encompassed the above method wherein the disease or condition is selected from the group consisting of: acute or chronic pain, osteoarthritis, rheumatoid arthritis, bursitis, ankylosing sponylitis and primary dysmenorrhea.

The following compounds exemplify the invention. These compounds were synthesized following the schemes and examples described below.

TABLE 1

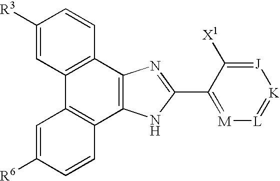

| Ex | R³/R⁶ | R⁶/R³ | X¹ |
|---|---|---|---|
| 1 | H | H | H |
| 2 | H | H | H |
| 3 | H | H | Cl |
| 4 | H | H | H |
| 5 | H | H | Br |
| 6 | H | H | CF₃ |
| 7 | H | H | Cl |
| 8 | H | H | H |
| 9 | H | H | Cl |
| 10 | H | H | H |
| 11 | H | H | CH₃ |
| 12 | H | H | F |
| 13 | H | H | Ph |
| 14 | H | H | H |
| 15 | H | H | Cl |
| 16 | H | H | CH₃ |
| 17 | H | H | CF₃ |
| 18 | Br | Br | Cl |
| 19 | H | H | Br |
| 20 | Br | CO₂H | Cl |
| 21 | H | H | Br |
| 22 | Br | H | Cl |
| 23 | CO₂CH₃ | CO₂CH₃ | Cl |
| 24 | H | H | 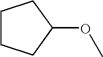 |
| 25 | Br | Br | H |
| 26 | H | H | H |
| 27 | Br | Br | H |
| 28 | H | H | H |
| 29 | H | H | OCH₃ |
| 30 | H | H | 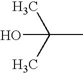 |
| 31 | H | H | H |
| 32 | Br | Br | CH₃ |
| 33 | Br | Br | 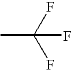 |
| 34 | H | H | 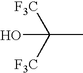 |
| 35 | Br | Br | H |
| 36 | H | H | H |
| 37 | Br | Br | H |
| 38 | H | H | H |
| 39 | Cl | Br | F |
| 40 | 3-pyridyl | 3-pyridyl | Cl |
| 41 | Br | 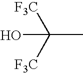 | Cl |
| 42 | Cl | Br | F |
| 43 | H | H | H |
| 44 | H | H | Cl |
| 45 | Br | Br | H |
| 46 | Br | Br | CF₃ |
| 47 | Br | Br | H |
| 48 | Br | Br | CH₃ |
| 49 | Br | Br | H |
| 50 | H | H | H |
| 51 | Br | Br | CF₃ |
| 52 | CN | CN | H |
| 53 | CN | Br | Cl |
| 54 | Br | Br | H |
| 55 | H | 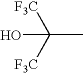 | Cl |
| 56 | Br | H | Cl |
| 57 | CN | Br | 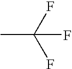 |
| 58 | CH₃ | Br | Cl |
| 59 | OCH₃ | Br | Cl |
| 60 | Br | Br | Cl |
| 61 | H | CO₂H | Cl |
| 62 | H | H | I |
| 63 | 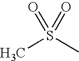 | Br | Cl |
| 64 | H | 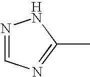 | Cl |
| 65 | H | 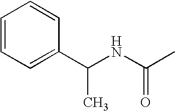 | Cl |
| 66 | Br | 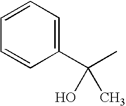 | Cl |
| 67 | 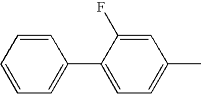 | H | Cl |
| 68 | Br | 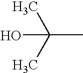 | Cl |
| 69 | Br | 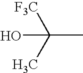 | Cl |
| 70 | H | H | H |
| 71 | H | H | Cl |
| 72 | H | H | F |
| 73 | H | H | F |
| 74 | H | H | F |
| 75 | H | H | F |
| 76 | 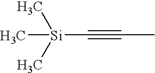 | H | F |
| 77 | 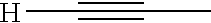 | H | F |
| 78 | i-Pr | Br | Cl |
| 79 | 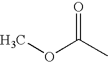 | 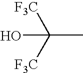 | Cl |
| 80 | 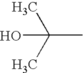 | 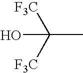 | Cl |
| 81 | CH₃ | H | Cl |
| 82 | CH₃ | 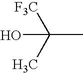 | Cl |
| 83 | CH₃ | CH₃ | Cl |
| 84 | 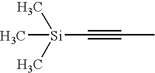 | 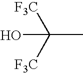 | F |
| 85 | 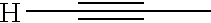 | 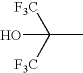 | F |
| 86 | 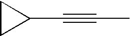 | 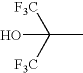 | F |
| 87 | 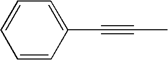 | 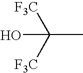 | F |
| 88 | CHO | 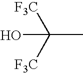 | Cl |
| 89 | 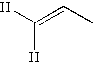 | 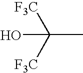 | F |
| 90 | 3-pyridyl | 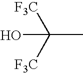 | Cl |
| 91 | Br | 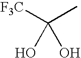 | Cl |
| 92 | 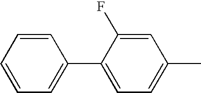 | 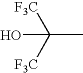 | Cl |
| 93 | 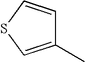 | 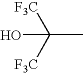 | Cl |
| 94 | (CH₃)₃Si— | 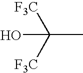 | Cl |
| 95 | Br | H | CF₃ |
| 96 | Br | 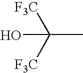 | CF₃ |
| 97 | OCH₃ | 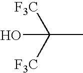 | Cl |
| 98 | 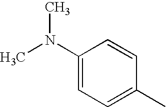 | 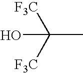 | Cl |
| 99 | I | 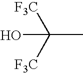 | F |
| 100 | 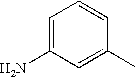 | 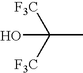 | Cl |
| 101 | 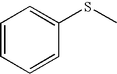 | 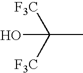 | F |
| 102 | 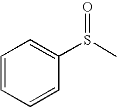 | 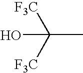 | F |
| 103 | 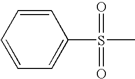 | 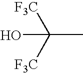 | F |
| 104 | 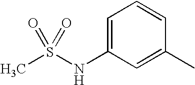 | 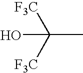 | Cl |
| 105 | 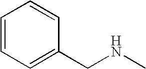 | 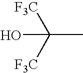 | Cl |
| 106 | Br | 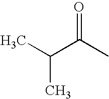 | Cl |
| 107 | Br | 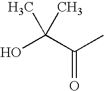 | Cl |
| 108 | Br | 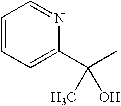 | Cl |
| 109 | 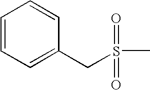 | 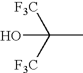 | F |
| 110 | 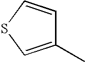 | 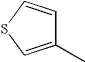 | F |
| 111 | F | H | Cl |
| 112 | 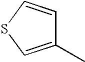 | Br | F |
| 113 | 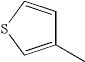 | 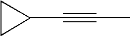 | F |
| 114 | 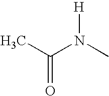 | H | Cl |
| 115 | 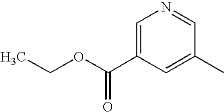 | 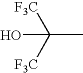 | Cl |
| 116 | 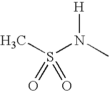 | H | Cl |
| 117 | —SO₂NH₂ | H | F |
| 118 | 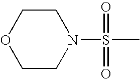 | H | F |
| 119 | 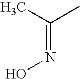 | Br | Cl |
| 120 | 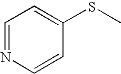 | 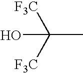 | F |
| 121 | 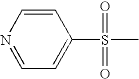 | 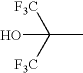 | F |
| 122 | 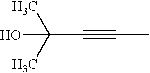 | 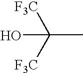 | F |
| 123 | 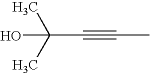 | Br | F |
| 124 | 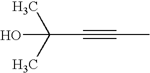 | 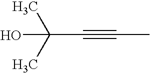 | F |
| 125 | Cl | H | Cl |
| 126 | Br | 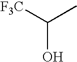 | F |
| 127 | 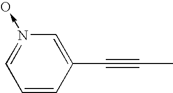 | 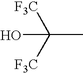 | F |
| 128 | 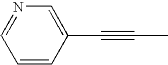 | 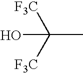 | F |
| 129 | H | H | H |
| 130 | H | H | OH |
| 131 | H | H | H |
| 132 | 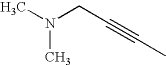 | 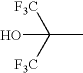 | F |
| 133 | 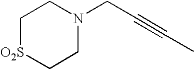 | 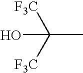 | F |
| 134 | H | H | OH |
| 135 | H | H | OCH₃ |
| 136 | Br | Br | Br |
| 137 | Cl | Br | Cl |
| 138 | Cl | 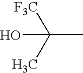 | Cl |
| 139 | CN | H | F |
| 140 | CN | 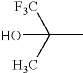 | Cl |
| 141 | Br | Br | I |
| 142 | Cl | 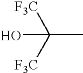 | Cl |
| 143 | H | H | 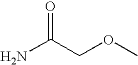 |
| 144 | Br | Br | 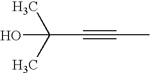 |
| 145 | Cl | 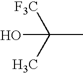 | H |
| 146 | OCH₃ | H | Cl |
| 147 | H | H | Br |
| 148 | H | H | F |
| 149 | Cl | H | Cl |
| 150 | Br | 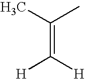 | Cl |
| 151 | Cl | H | 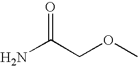 |
| 152 | Cl | Br | Br |
| 153 | 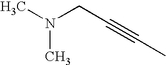 | Br | Cl |
| 154 | 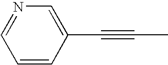 | Br | Cl |
| 155 | Br | 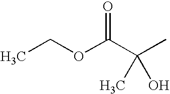 | Cl |
| 156 | 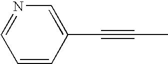 | 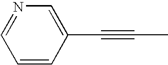 | Cl |
| 157 | Cl | Cl | Cl |
| 158 | H | H | Br |
| 159 | H | H | F |
| 160 | Br | 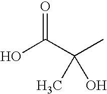 | Cl |
| 161 | 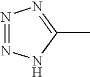 | Cl | Cl |
| 162 | 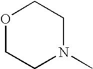 | H | Cl |
| 163 | Br | OH | Cl |
| 164 | 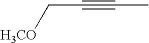 | Br | Cl |
| 165 | 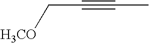 | 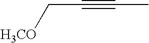 | Cl |
| 166 | 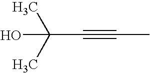 | H | Cl |
| 167 | Cl | H | Cl |
| 168 | Cl | 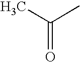 | Cl |
| 169 | 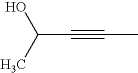 | Br | Cl |
| 170 | 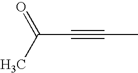 | Br | Cl |
| 171 | 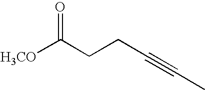 | Br | Cl |
| 172 | 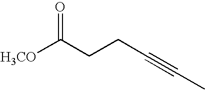 | 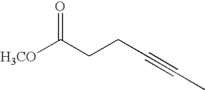 | Cl |
| 173 | H | H | Cl |
| 174 | 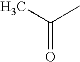 | 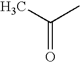 | Cl |
| 175 | 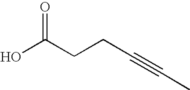 | Br | Cl |
| 176 | H | H | H |
| 177 | Cl | Br | Cl |
| 178 | Br | 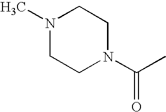 | Cl |
| 179 | Cl | H | Br |
| 180 | Cl | 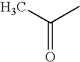 | Cl |
| 181 | H | H | H |
| 182 | Cl | H | F |
| 183 | Br | 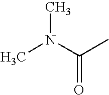 | Cl |
| 184 | Br | 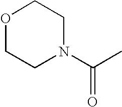 | Cl |
| 185 | I | I | I |
| 186 | Br | NH₂ | Cl |
| 187 | Br | 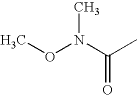 | Cl |
| 188 | Br | 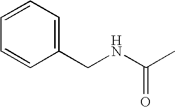 | Cl |
| 189 | H | H | Cl |
| 190 | 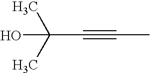 | 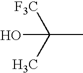 | Cl |
| 191 | H | H | Cl |
| 192 | 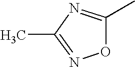 | Br | Cl |
| 193 | Br | Cl | Br |
| 194 | Br | 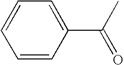 | Cl |
| 195 | Cl | 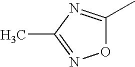 | Cl |
| 196 | Cl | 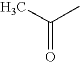 | I |
| 197 | Cl | 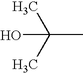 | I |
| 198 | Cl | H | Cl |
| 199 | Cl | 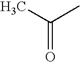 | Br |
| 200 | Cl | 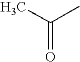 | I |
| 201 | Cl | 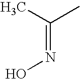 | I |
| 202 | Cl | 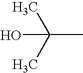 | I |
| 203 | I | I | I |
| 204 | Br | 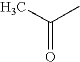 | Cl |
| 205 | I | 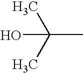 | Cl |
| 206 | Cl | 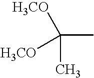 | I |
| 207 | Cl | 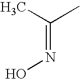 | Br |
| 208 | Cl | 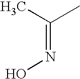 | Cl |
| 209 | Cl | 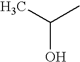 | Br |
| 210 | 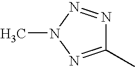 | Cl | Br |
| 211 | Cl | 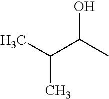 | Cl |
| 212 | Cl | 3-pyridyl | Cl |
| 213 | Cl | 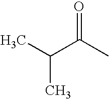 | Cl |
| 214 | 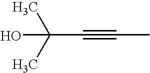 | Cl | Cl |
| 215 | 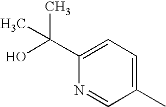 | Cl | Cl |
| 216 | Cl | 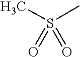 | Br |
| 217 | Br | H | Cl |
| 218 | Br | H | Br |
| 219 | I | H | I |
| 220 | Cl | 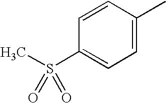 | Br |
| 221 | Cl | 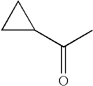 | Br |
| 222 | Cl | 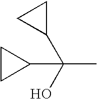 | Br |
| 223 | Cl | 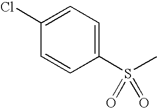 | Br |
| 224 | Cl | F | Br |
| 225 | 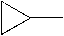 | H | Br |
| 226 | Cl | 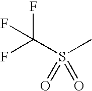 | Br |
| 227 | Cl | 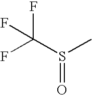 | Br |
| 228 | Cl | 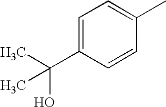 | Br |
| 229 | Cl | 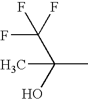 | Br |
| 230 | Br | 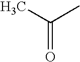 | Br |
| 231 | Br | 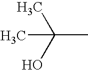 | Br |
| 232 | 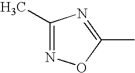 | Br | Br |
| 233 | Cl | Br | Br |
| 234 | Br | Cl | Br |
| 235 | Br | 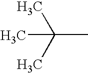 | Br |
| 236 | 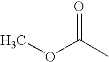 | 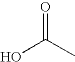 | Br |

| Ex | J | K | L | M |
|---|---|---|---|---|
| 1 | CH | CCO₂CH₃ | CH | CH |
| 2 | Cn-Pr | 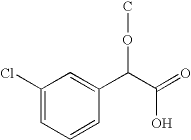 | Cn-Pr | CH |
| 3 | CH | CH | CH | CF |
| 4 | H | H | 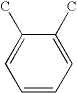 | |
| 5 | CH | CH | CH | CH |
| 6 | CH | CH | CH | CH |
| 7 | CH | CH | CH | CH |
| 8 | CH | Ct-Bu | CH | CH |
| 9 | CH | CCl | CH | CH |
| 10 | CH | CH | CF | CH |
| 11 | CH | CH | CH | CH |
| 12 | CH | CH | CH | CH |
| 13 | CH | CH | CH | CH |
| 14 | CH | CBr | CH | CH |
| 15 | CH | N | CH | CCl |
| 16 | CH | CCH₃ | CH | CCH₃ |
| 17 | CH | CF | CH | CH |
| 18 | CH | CH | CH | CF |
| 19 | CH | CH | CH | N |
| 20 | CH | CH | CH | CF |
| 21 | CH | N | CH | CH |
| 22 | CH | CH | CH | CF |
| 23 | CH | CH | CH | CF |
| 24 | CH | CH | CH | CH |
| 25 | CH | CH | CH | C(CO₂CH₃) |
| 26 | CH | CH | N | CCl |
| 27 | CH | CH | CH | C(CO₂Na) |
| 28 | C(NO₂) | CH | CH | CCl |
| 29 | CH | CH | CH | CH |
| 30 | CH | CH | CH | CH |
| 31 | C(SCH₃) | CH | CH | CCH₃ |
| 32 | CH | CH | CH | CH |
| 33 | CH | CF | CH | CH |
| 34 | CH | CH | CH | CH |
| 35 | CH | CH | N | CCl |
| 36 | C(SO₂CH₃) | CH | CH | CCl |
| 37 | CH | CH | CH | CNO₂ |
| 38 | C(OCH₃) | CH | CH | CBr |
| 39 | CH | CH | CH | C(CONH₂) |
| 40 | CH | CH | CH | CF |
| 41 | CH | CH | CH | CF |
| 42 | CH | CH | CH | C(COCH₃) |
| 43 | CH | CCl | CH | CH |
| 44 | 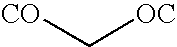 | | CH | CH |
| 45 | CH | CH(C(SCF₃) | CH | CH |
| 46 | CH | CH | CH | CH |
| 47 | CH | CH | CH | Cl |
| 48 | CH | CCH₃ | CH | CH |
| 49 | CH | CH | CH | CBr |
| 50 | CH | CH | CH | Cl |
| 51 | CH | CH | CH | CF |
| 52 | CH | CF | CH | C(CF₃) |
| 53 | CH | CF | CH | CH |
| 54 | CH | C(N(CH₃)₂) | CH | CCH₃ |
| 55 | CH | CH | CH | CF |
| 56 | CH | CF | CH | CH |
| 57 | CH | CF | CH | CH |
| 58 | CH | CH | CH | CF |
| 59 | CH | CH | CH | CF |
| 60 | CH | CF | CH | CH |
| 61 | CH | CH | CH | CF |
| 62 | CH | CH | CH | CF |
| 63 | CH | CH | CH | CF |
| 64 | CH | CH | CH | CF |
| 65 | CH | CH | CH | CF |
| 66 | CH | CH | CH | CF |
| 67 | CH | CH | CH | CF |
| 68 | CH | CH | CH | CF |
| 69 | CH | CH | CH | CF |
| 70 | CH | CH | CH | CH |
| 71 | CH | CH | CH | CCl |
| 72 | CH | CH | CH | CF |
| 73 | CF | CH | CH | CH |
| 74 | CH | CH | CF | CH |
| 75 | CF | CH | CH | CF |
| 76 | CH | CH | CH | CCl |
| 77 | CH | CH | CH | CCl |
| 78 | CH | CH | CH | CF |
| 79 | CH | CH | CH | CF |
| 80 | CH | CH | CH | CF |
| 81 | CH | CH | CH | CF |
| 82 | CH | CH | CH | CF |
| 83 | CH | CH | CH | CF |
| 84 | CH | CH | CH | CCl |
| 85 | CH | CH | CH | CCl |
| 86 | CH | CH | CH | CCl |
| 87 | CH | CH | CH | CCl |
| 88 | CH | CH | CH | CF |
| 89 | CH | CH | CH | CCl |
| 90 | CH | CH | CH | CF |
| 91 | CH | CH | CH | CF |
| 92 | CH | CH | CH | CF |
| 93 | CH | CH | CH | CF |
| 94 | CH | CH | CH | CF |
| 95 | CH | CH | CH | CF |
| 96 | CH | CH | CH | CF |
| 97 | CH | CH | CH | CF |
| 98 | CH | CH | CH | CF |
| 99 | CH | CH | CH | CCl |
| 100 | CH | CH | CH | CF |
| 101 | CH | CH | CH | CCl |
| 102 | CH | CH | CH | CCl |
| 103 | CH | CH | CH | CCl |
| 104 | CH | CH | CH | CF |
| 105 | CH | CH | CH | CF |
| 106 | CH | CH | CH | CF |
| 107 | CH | CH | CH | CF |
| 108 | CH | CH | CH | CF |
| 109 | CH | CH | CH | CCl |
| 110 | CH | CH | CH | CCl |
| 111 | CH | CH | CH | CF |
| 112 | CH | CH | CH | CCl |
| 113 | CH | CH | CH | CCl |
| 114 | CH | CH | CH | CF |
| 115 | CH | CH | CH | CF |
| 116 | CH | CH | CH | CF |
| 117 | CH | CH | CH | CCl |
| 118 | CH | CH | CH | CCl |
| 119 | CH | CH | CH | CF |
| 120 | CH | CH | CH | CCl |
| 121 | CH | CH | CH | CCl |
| 122 | CH | CH | CH | CCl |
| 123 | CH | CH | CH | CCl |
| 124 | CH | CH | CH | CCl |
| 125 | CH | CH | CH | CF |
| 126 | CH | CH | CH | CCl |
| 127 | CH | CH | CH | CCl |
| 128 | CH | CH | CH | CCl |
| 129 | CH | CH | CH | N |
| 130 | CH | CH | CH | CH |
| 131 | CH | CH | CH | NO |
| 132 | CH | CH | CH | CCl |
| 133 | CH | CH | CH | CCl |
| 134 | CH | COH | CH | CH |
| 135 | CH | CH | CH | C(OCH₃) |
| 136 | CH | CH | CH | CF |
| 137 | CH | CH | CH | CF |
| 138 | CH | CH | CH | CF |
| 139 | CH | CH | CH | CCl |
| 140 | CH | CH | CH | CF |
| 141 | CH | CH | CH | CF |
| 142 | CH | CH | CH | CF |
| 143 | CH | CH | CH | CH |
| 144 | CH | CH | CH | CF |
| 145 | CH | CH | CH | CF |
| 146 | CH | CH | CH | CF |
| 147 | CH | CH | CH | CBr |
| 148 | CH | CH | CH | CBr |
| 149 | CH | CH | CH | COH |
| 150 | CH | CH | CH | CF |
| 151 | CH | CH | CH | CCl |
| 152 | CH | CH | CH | CF |
| 153 | CH | CH | CH | CF |
| 154 | CH | CH | CH | CF |
| 155 | CH | CH | CH | CF |
| 156 | CH | CH | CH | CF |
| 157 | CH | CH | CH | CF |
| 158 | CH | N | CH | CBr |
| 159 | CH | N | CH | CH |
| 160 | CH | CH | CH | CF |
| 161 | CH | CH | CH | CF |
| 162 | CH | CH | CH | CF |
| 163 | CH | CH | CH | CF |
| 164 | CH | CH | CH | CF |
| 165 | CH | CH | CH | CF |
| 166 | CH | CH | CH | CF |
| 167 | CH | N | CH | CCl |
| 168 | CH | CH | CH | CF |
| 169 | CH | CH | CH | CF |
| 170 | CH | CH | CH | CF |
| 171 | CH | CH | CH | CF |
| 172 | CH | CH | CH | CF |
| 173 | N | CH | CH | N |
| 174 | CH | CH | CH | CF |
| 175 | CH | CH | CH | CF |
| 176 | CH | CH | N | CBr |
| 177 | CH | CH | CH | CCl |
| 178 | CH | CH | CH | CF |
| 179 | CH | N | CH | CBr |
| 180 | CH | CH | CH | CCl |
| 181 | N | CH | CH | CBr |
| 182 | CH | N | CH | CF |
| 183 | CH | CH | CH | CF |
| 184 | CH | CH | CH | CF |
| 185 | CH | CH | CH | CF |
| 186 | CH | CH | CH | CF |
| 187 | CH | CH | CH | CF |
| 188 | CH | CH | CH | CF |
| 189 | CH | CH | CH | N |
| 190 | CH | CH | CH | CF |
| 191 | N | CH | CH | CCl |
| 192 | CH | CH | CH | CF |
| 193 | CH | CH | CH | CBr |
| 194 | CH | CH | CH | CF |
| 195 | CH | CH | CH | CF |
| 196 | CH | CH | CH | CF |
| 197 | CH | CH | CH | CF |
| 198 | N | CH | CH | CCl |
| 199 | CH | CH | CH | CBr |
| 200 | CH | CH | CH | CI |
| 201 | CH | CH | CH | CI |
| 202 | CH | CH | CH | CI |
| 203 | CH | CH | CH | CI |
| 204 | CH | CH | CH | CF |
| 205 | CH | CH | CH | CF |
| 206 | CH | CH | CH | CI |
| 207 | CH | CH | CH | CBr |
| 208 | CH | CH | CH | CCl |
| 209 | CH | CH | CH | CBr |
| 210 | CH | CH | CH | CBr |
| 211 | CH | CH | CH | CCl |
| 212 | CH | CH | CH | CCl |
| 213 | CH | CH | CH | CCl |
| 214 | CH | CH | CH | CCl |
| 215 | CH | CH | CH | CCl |
| 216 | CH | CH | CH | CBr |
| 217 | CH | CH | CH | CCl |
| 218 | CH | CH | CH | CBr |
| 219 | CH | CH | CH | CI |
| 220 | CH | CH | CH | CBr |
| 221 | CH | CH | CH | CBr |
| 222 | CH | CH | CH | CBr |
| 223 | CH | CH | CH | CBr |
| 224 | CH | CH | CH | CBr |
| 225 | CH | CH | CH | CBr |
| 226 | CH | CH | CH | CBr |
| 227 | CH | CH | CH | CBr |
| 228 | CH | CH | CH | CBr |
| 229 | CH | CH | CH | CBr |
| 230 | CH | CH | CH | CBr |
| 231 | CH | CH | CH | CBr |
| 232 | CH | CH | CH | CBr |
| 233 | CH | N | CH | Br |
| 234 | CH | COH | CH | CBr |
| 235 | CH | CH | CH | CBr |
| 236 | CH | CH | CH | CBr |

TABLE 2

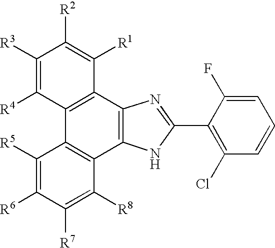

| Ex | R¹ | R² | R³ | R⁴ | R⁵ | R⁶ | R⁷ | R⁸ |
|---|---|---|---|---|---|---|---|---|
| 238 | Br | H | H | H | H | H | H | |
| 239 | H | Br | H | H | H | H | H | H |
| 240 | H | H | H | Br | H | H | H | H |
| 241 | F | H | F | H | H | H | H | H |
| 242 | H | H | H | 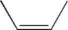 | | H | H | H |
| 243 | H | Cl | H | H | H | H | H | H |
| 244 | H | Cl | H | H | H | H | | H |
| 245 | H | Cl | H | H | H | CH₃ | H | H |
| 246 | H | Cl | H | H | H | H | H | CH₃ |
| 247 | H | Cl | H | H | H | H | 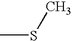 | H |
| 248 | H | Cl | H | H | H | H | 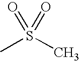 | H |
| 249 | H | H | H | H | F | H | F | H |
| 250 | H | H | H | H | 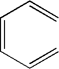 | | H | H |
| 251 | H | H | H | H | H | CH₃ | F | H |
| 252 | H | H | H | H | H | H | 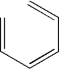 | |
| 253 | H | Cl | Cl | H | H | Br | H | H |
| 254 | H | H | Cl | Cl | H | Br | H | H |
| 255 | H | F | F | F | H | Br | H | H |

The invention includes, as appropriate, pharmaceutically acceptable salts of any of the aforementioned compounds. For purposes of this specification, the heading “R³/R⁶” means that the substituent indicated in that column is substituted at the position represented by either R³ or R⁶. In the adjacent column, the heading “R⁶/R³” means the indicated substituent is substituted at the position R³ or R⁶ not substituted in the previous column. By way of example, Example 6 represents R³═CN and R⁶═H or R³═H and R⁶═CN, representing both tautomers.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl and 1,1-dimethylethyl.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₃₋₆alkynyl, for example, includes, propenyl, 1-methylethenyl, butenyl and the like.

The term “alkoxy” means alkoxy groups of a straight, branched or cyclic configuration having the indicated number of carbon atoms. C₁₋₆alkoxy, for example, includes methoxy, ethoxy, propoxy, isopropoxy, and the like.

The term “cycloalkyl” means mono-, bi- or tri-cyclic structures, optionally combined with linear or branched structures, having the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl, cyclobutylmethyl cyclopropylmethyl and the like.

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I is shown without a definitive stereochemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. The compound of Formula I exists in the following tautomeric forms:

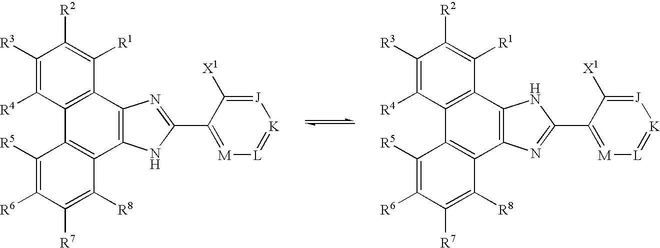

The individual tautomers as well as mixture thereof are encompassed within Formula I.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu. Exemplifying prodrugs of the invention are compounds of Formula C.

The term “treating a microsomal prostaglandin E synthase-1 mediated disease or condition” means treating or preventing any disease or condition that is advantageously treated or prevented by inhibiting the microsomal prostaglandin E synthase-1 (mPGES-1) enzyme. The term includes the relief of pain, fever and inflammation of a variety of conditions including rheumatic fever, symptoms associated with influenza or other viral infections, common cold, low back and neck pain, dysmenorrhea, headache, migraine (acute and prophylactic treatment), toothache, sprains and strains, myositis, neuralgia, synovitis, arthritis, including rheumatoid arthritis, degenerative joint diseases (osteoarthritis), gout and ankylosing spondylitis, acute, subacute and chronic musculoskeletal pain syndromes such as bursitis, burns, injuries, and pain following surgical and dental procedures as well as the preemptive treatment of surgical pain. In addition, the term includes the inhibition cellular neoplastic transformations and metastic tumor growth and hence the treatment of cancer. The term also includes the treatment of endometriosis and Parkinson's disease as well as the treatment of mPGES-1 mediated proliferative disorders such as may occur in diabetic retinopathy and tumor angiogenesis. The term “treating” encompasses not only treating a patient to relieve the patient of the signs and symptoms of the disease or condition but also prophylactically treating an asymptomatic patient to prevent the onset or progression of the disease or condition.

The term “amounts that are effective to treat” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician. Suitable dosage levels of the compound of Formula I used in the present invention are described below. The compound may be administered on a regimen of once or twice per day.

The pharmaceutical compositions of the present invention comprise a compound of Formula I as an active ingredient or a pharmaceutically acceptable salt, thereof, and may also contain a pharmaceutically acceptable carrier and optionally other therapeutic ingredients. The term “pharmaceutically acceptable salts” include salts prepared from bases that result in non-toxic pharmaceutically acceptable salts, including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from acids that result in pharmaceutically acceptable salts, including inorganic and organic acids. Such acids include acetic, adipic, aspartic, 1,5-naphthalenedisulfonic, benzenesulfonic, benzoic, camphorsulfonic, citric, 1,2-ethanedisulfonic, ethanesulfonic, ethylenediaminetetraacetic, fumaric, glucoheptonic, gluconic, glutamic, hydriodic, hydrobromic, hydrochloric, isethionic, lactic, maleic, malic, mandelic, methanesulfonic, mucic, 2-naphthalenesulfonic, nitric, oxalic, pamoic, pantothenic, phosphoric, pivalic, propionic, salicylic, stearic, succinic, sulfuric, tartaric, p-toluenesulfonic acid, undecanoic, 10-undecenoic, and the like.

By virtue of the mPGES-1 inhibitory activity of compounds of the present invention, the compounds of Formula I are useful for the relief of pain, fever and inflammation of a variety of conditions including rheumatic fever, symptoms associated with influenza or other viral infections, common cold, low back and neck pain, dysmenorrhea, headache, migraine (acute and prophylactic treatment), toothache, sprains and strains, myositis, neuralgia, synovitis, arthritis, including rheumatoid arthritis, juvenile rheumatoid arthritis, degenerative joint diseases (osteoarthritis), acute gout and ankylosing spondylitis, acute, subacute and chronic musculoskeletal pain syndromes such as bursitis, burns, injuries, and pain following surgical and dental procedures as well as the preemptive treatment of surgical pain. In addition, such a compound may inhibit cellular neoplastic transformations and metastic tumor growth and hence can be used in the treatment of cancer. Compounds of Formula I may also be useful for the treatment or prevention of endometriosis, hemophilic arthropathy and Parkinson's disease.

Compounds of Formula I will also inhibit prostanoid-induced smooth muscle contraction by preventing the synthesis of contractile prostanoids and hence may be of use in the treatment of dysmenorrhea, premature labor and asthma.

By virtue of their selective inhibition of the mPGES-1 enzyme, the compounds of Formula I will prove useful as an alternative to conventional non-steroidal antiinflammatory drugs (NSAID'S) particularly where such non-steroidal antiinflammatory drugs may be contra-indicated such as in patients with peptic ulcers, gastritis, regional enteritis, ulcerative colitis, diverticulitis or with a recurrent history of gastrointestinal lesions; GI bleeding, coagulation disorders including anemia such as hypoprothrombinemia, haemophilia or other bleeding problems (including those relating to reduced or impaired platelet function); kidney disease (e.g., impaired renal function); those prior to surgery or taking anticoagulants; and those susceptible to NSAID induced asthma.

The compounds of the invention are also useful for treating or preventing a neoplasia in a subject in need of such treatment or prevention. The term “treatment” includes partial or total inhibition of the neoplasia growth, spreading or metastasis, as well as partial or total destruction of the neoplastic cells. The term “prevention” includes either preventing the onset of clinically evident neoplasia altogether or preventing the onset of a preclinically evident stage of neoplasia in individuals at risk. Also intended to be encompassed by this definition is the prevention of initiation for malignant cells or to arrest or reverse the progression of premalignant cells to malignant cells. This includes prophylactic treatment of those at risk of developing the neoplasia. The term “subject” for purposes of treatment includes any human or mammal subject who has any one of the known neoplasias, and preferably is a human subject. For methods of prevention, the subject is any human or animal subject, and preferably is a human subject who is at risk for obtaining a neoplasia. The subject may be at risk due to exposure to carcinogenic agents, being genetically predisposed to have the neoplasia, and the like.

The term “neoplasia” includes both benign and cancerous tumors, growths and polyps. Thus, the compounds of the invention are useful for treating or preventing benign tumors, growths and polyps including squamous cell papilloma, basal cell tumor, transitional cell papilloma, adenoma, gastrinoma, cholangiocellular adenoma, hepatocellular adenoma, renal tubular adenoma, oncocytoma, glomus tumor, melanocytic nevus, fibroma, myxoma, lipoma, leiomyoma, rhabdomyoma, benign teratoma, hemangioma, osteoma, chondroma and meningioma. The compounds of the invention are also useful for treating or preventing cancerous tumors, growths and polyps including squamous cell carcinoma, basal cell carcinoma, transitional cell carcinoma, adenocarcinoma, malignant gastrinoma, cholangiocelleular carcinoma, hepatocellular carcinoma, renal cell carcinoma, malignant melanoma, fibrosarcoma, myxosarcoma, liposarcoma, leimyosarcoma, rhabdomyosarcoma, malignant teratoma, hemangiosarcoma, Kaposi sarcoma, lymphangiosarcoma, ostreosarcoma, chondrosarcoma, malignant meningioma, non-Hodgkin lymphoma, Hodgkin lymphoma and leukemia. For purposes of this specification, “neoplasia” includes brain cancer, bone cancer, epithelial cell-derived neoplasia (epithelial carcinoma), basal cell carcinoma, adenocarcinoma, gastrointestinal cancer such as lip cancer, mouth cancer, esophogeal cancer, small bowel cancer and stomach cancer, colon cancer, rectal cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that affect epithelial, mesenchymal or blood cells throughout the body. The compounds of the invention are useful for treating or preventing any of the aforementioned cancers. The compounds of the invention are useful for treating or preventing benign and cancerous tumors, growths and polyps of the following cell types: squamous epithelium, basal cells, transitional epithelium, glandular epithelium, G cells, bile ducts epithelium, hepatocytes, tubules epithelium, melanocytes, fibrous connective tissue, cardiac skeleton, adipose tissue, smooth muscle, skeletal muscle, germ cells, blood vessels, lymphatic vessels, bone, cartilage, meninges, lymphoid cells and hematopoietic cells. The compounds can be used to treat subjects having adenomatous polyps, including those with familial adenomatous polyposis (FAP). Additionally, the compounds can be used to prevent polyps from forming in patients at risk of FAP. Preferably, the compounds of the invention are useful for treating or preventing the following cancers: colorectal, esophagus stomach, breast, head and neck, skin, lung, liver, gall bladder, pancreas, bladder, endometrium cervix, prostate, thyroid and brain.

Similarly, compounds of Formula I will be useful as a partial or complete substitute for conventional NSAIDs in preparations wherein they are presently co-administered with other agents or ingredients. Thus in further aspects, the invention encompasses pharmaceutical compositions for treating mPGES-1 mediated diseases as defined above comprising a non-toxic therapeutically effective amount of the compound of Formula I as defined above and one or more ingredients such as another pain reliever including acetaminophen or phenacetin; opioid analgesics, such as codeine, fentanyl, hydromorphone, levorphanol, meperidine, methadone, morphine, oxycodone, oxymorphine, propoxyphene, buprenorphine, butorphanol, dezocine, nalbuphine and pentazocine; a potentiator including caffeine; an H2-antagonist; aluminum or magnesium hydroxide; simethicone; a decongestant including phenylephrine, phenylpropanolamine, pseudophedrine, oxymetazoline, ephinephrine, naphazoline, xylometazoline, propylhexedrine, or levo-desoxyephedrine; an antitussive including codeine, hydrocodone, caramiphen, carbetapentane, or dextramethorphan; a diuretic; a sedating or non-sedating antihistamine; a proton pump inhibitor, such as omeprazole; a bradykinin-1 antagonist; a VR1 receptor antagonist; and a sodium channel blocker (NAV1). For the treatment or prevention of migraine, the invention also encompasses co-administration with a 5-HT agonist such as rizatriptan, sumatriptan, zolmitriptan and naratriptan, or a CGRP antagonist. In addition the invention encompasses a method of treating mPGES-I mediated diseases comprising: administration to a patient in need of such treatment a non-toxic therapeutically effect amount of the compound of Formula I, optionally co-administered with one or more of such ingredients as listed immediately above.

As indicated above, pharmaceutical compositions for treating mPGES-1 mediated diseases as defined may optionally include one or more ingredients as listed above.

In another aspect, the invention encompasses co-administering a proton pump inhibitor with a compound of Formula I. The proton pump inhibitors that may be utilized in this aspect of the invention include omeprazole, lansoprazole, rabeprazole, pantoprazole, and esomeprazole, or a pharmaceutically acceptable salt of any of the aforementioned. These proton pump inhibitors are commercially available, e.g., omeprazole (PRILOSEC, AstraZeneca), lansoprazole (PREVACID, TAP Pharmaceuticals), rabeprazole (ACIPHEX, Janssen Pharmaceutica), pantoprazole (PROTONIX, Wyeth-Ayerst), and esomeprazole (NEXIUM, AstraZeneca). The said proton pump inhibitors may be administered at conventional doses. For example, omeprazole or omeprazole magnesium may be administered at a dose of 10 mg, 20 mg or 40 mg. Lansoprazole may be administered at a dose of 15 mg or 30 mg. Rabeprazole sodium may be administered at a dose of 20 mg. Pantoprazole may be administered at a dose of 20 mg or 40 mg. Esomeprazole may be administered at a dose of 20 mg or 40 mg. The compound of Formula I and the proton pump inhibitor may be administered concomitantly in a single pharmaceutical dosage form or as two separate dosage forms taken by a patient substantially at the same time. Alternatively, the compound of Formula I and the proton pump inhibitor may be taken sequentially at separately staggered times as long as the pharmaceutical effects of the two agents are being realized by the patient at the same time.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or an oil medium, for example peanut oil, liquid paraffin, or olive oil. Exemplifying a formulation for the present invention is a dry filled capsule containing a 50/50 blend of microcrystalline cellulose and lactose and 1 mg, 10 mg or 100 mg of the compound of Formula I.

Aqueous suspensions contain the active material in admixture with excipients suitable for the manufacture of aqueous suspensions. Such excipients are suspending agents, for example sodium carboxymethyl-cellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Liquid formulations include the use of self-emulsifying drug delivery systems and NanoCrystal® technology. Cyclodextrin inclusion complexes can also be utilized.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of Formula I are employed. (For purposes of this application, topical application shall include mouth washes and gargles.)

Pharmaceutical compositions of the invention may also utilize absorption enhancers such as tween 80, tween 20, Vitamin E TPGS (d-alpha-tocopheryl polyethylene glycol 1000 succinate) and Gelucire®.

Dosage levels of the order of from about 0.01 mg to about 140 mg/kg of body weight per day are useful in the treatment of the above-indicated conditions, or alternatively about 0.5 mg to about 7 g per patient per day. For example, inflammation may be effectively treated by the administration of from about 0.01 to 50 mg of the compound per kilogram of body weight per day, or alternatively about 0.5 mg to about 3.5 g per patient per day, preferably 2.5 mg to 1 g per patient per day.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg. Dosage amounts of 4 mg, 8 mg, 18 mg, 20 mg, 36 mg, 40 mg, 80 mg, 160 mg, 320 mg and 640 mg may also be employed. Dosage unit forms containing 1, 10 or 100 mg are also encompassed.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

### Methods of Synthesis

The compounds of Formula I of the present invention can be prepared according to the synthetic routes outlined in Schemes 1 to 4 below and by following the methods described therein. The imidazole of Formula I may be prepared from the requisite phenanthrenequinone i. The phenanthrene imidazole Ia is obtained by treating the phenanthrenequinone i and an appropriately substituted aldehyde ii with a reagent such as NH₄OAc or NH₄HCO₃ in a solvent such as acetic acid. Subsequent functional group interconversion can be done at any of the R¹ to R⁸ positions. For example, if one or more of the R¹ to R⁸ substituents equal Cl, Br or I and if X¹ is different from Br, Cl or I and if J, K, L and M are different from CBr, CCl or CI, Ia could be converted to Ib by placing Ia in the presence of a monosubstituted alkynyl, a stannane, a boronic acid, a borane or a boronate under conditions that promote cross coupling reactions, such as heating in the presence of a catalyst, such as Pd(PPh₃)₄ and CuI, in the presence of a base, such as sodium carbonate or diisopropylamine, and in an suitable solvent, such as THF, DMF or DME. Additional examples include, but are not limited to, functional group interconversion of one or more of the R¹ to R⁸ substituents and/or X¹ to heterocycles, amine functionalities, thiol moieties or other functional groups obtained after metallation followed by quenching with the appropriate electrophile. Additionally, if J, K, L or M equal nitrogen, this group can be oxidized with an oxidizing agent such as MCPBA or oxone, in a suitable solvent such as CH₂Cl₂. These last exemplified steps, or any other appropriate functional group transformation, can be iteratively repeated on R¹ to R⁸, X¹, J, K, L or M.

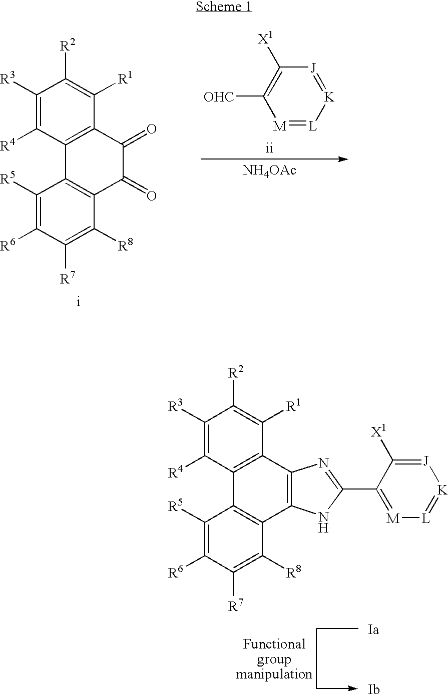

Phenanthrenequinone i can be prepared according to the sequences outlined in Schemes 2 to 4. As shown in Scheme 2, commercially available phenanthrenes iiia can be directly oxidized with an oxidizing agent, such as CrO₃, in a suitable solvent, such as acetic acid, to provide the phenanthrenequinone ia, or optionally, phenanthrene iiia could be further elaborated to phenanthrene iiib by the appropriate interconversion of any of the functional groups R¹ to R⁸, such as conversion of a methyl ketone to a halogen through a series of functional group transformations. Phenanthrene iiib can then be oxidized to phenanthrenequinone ia as described above. In addition, phenanthrenequinone ia can be further elaborated to phenanthrenequinone ib via synthetic sequences such as bromination with a brominating agent such as bromine in a solvent such as nitrobenzene, in the presence of an initiator such as benzoyl peroxide or AIBN.

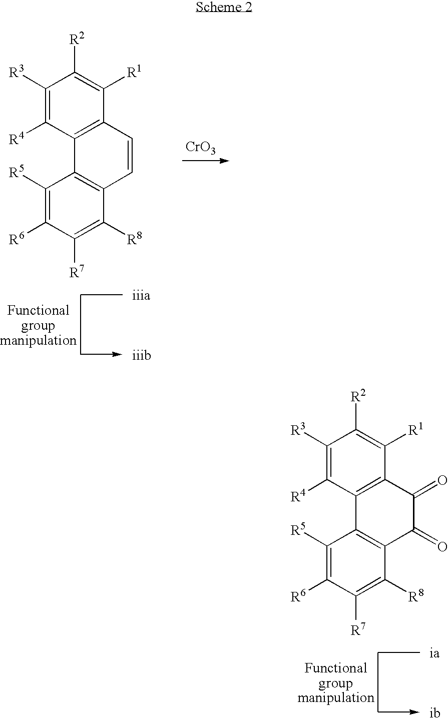

Alternatively, phenanthrenequinone i can be prepared as outlined in Scheme 3. Deprotonation of the phosphonium salt iv in the presence of a base, such as sodium hydride or sodium methoxide, in a solvent such as DMF, followed by the addition of the aldehyde v produces the stillbene vi as a mixture of E and Z isomers. Intramolecular cyclisation of this mixture upon exposition to UV light in the presence of an oxidizing agent, such as iodine, and an acid scavenger, such as propylene oxide, in a suitable solvent such as cyclohexane produces the phenanthrene viia. This phenanthrene viia can be directly oxidized with an oxidizing agent, such as CrO₃, in a suitable solvent, such as acetic acid, to provide the phenanthrenequinone i, or optionally, phenanthrene viia could be further elaborated to phenanthrene viib by the appropriate interconversion of any of the functional groups R¹ to R⁸, such as transmetallation with an organometallic reagent, such as butyl lithium, in a suitable solvent such as THF, followed by the addition of an electrophile, such as iodine or hexafluoroacetone. Phenanthrene viib can then be oxidized to phenanthrenequinone i as described above

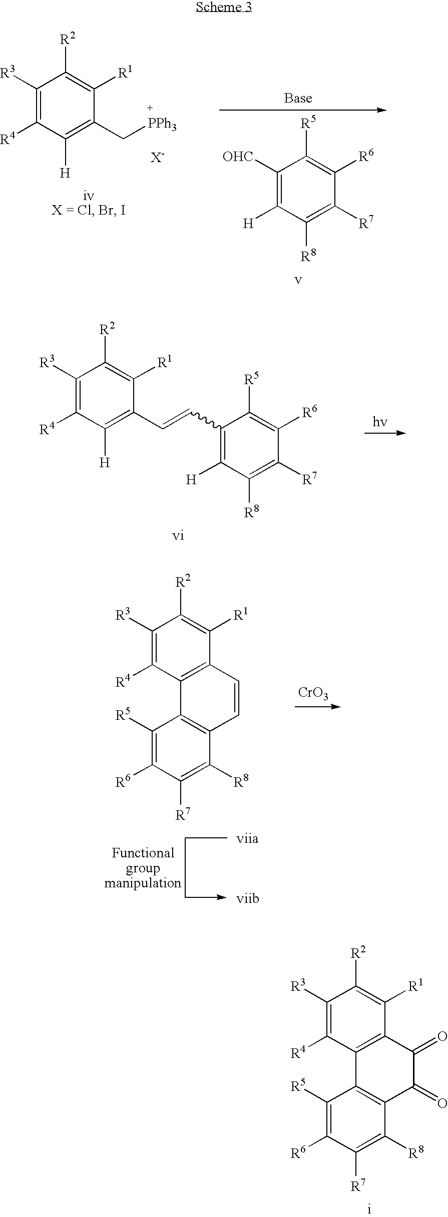

Scheme 4 describes an alternate route for the synthesis for phenanthrenequinone i. Phenylacetic acid viii can be condensed with the aldehyde ix in the presence of a base, such as potassium carbonate, and in the presence of acetic anhydride to afford the nitro stillbene x. This nitro aryl x is then reduced with an appropriate reducing agent, such as iron or iron sulfate, in the presence of ammonium hydroxide in a suitable solvent, such as acetic acid, to produce the aryl amine xi. Diazotization of this amine xi with sodium nitrite in the presence of aqueous hydroxide, such as sodium hydroxide, followed by acidification with an acid, such as sulfuric acid and sulfamic acid, and cyclisation in the presence of a catalyst, such as copper or ferrocene, generates the phenanthrene carboxylic acid xii. This phenanthrene carboxylic acid xii can be oxidized with simultaneous decarboxylation using an appropriate oxidizing agent, such as chromium trioxide in a suitable solvent, such as acetic acid, to afford the phenanthrenequinone i.

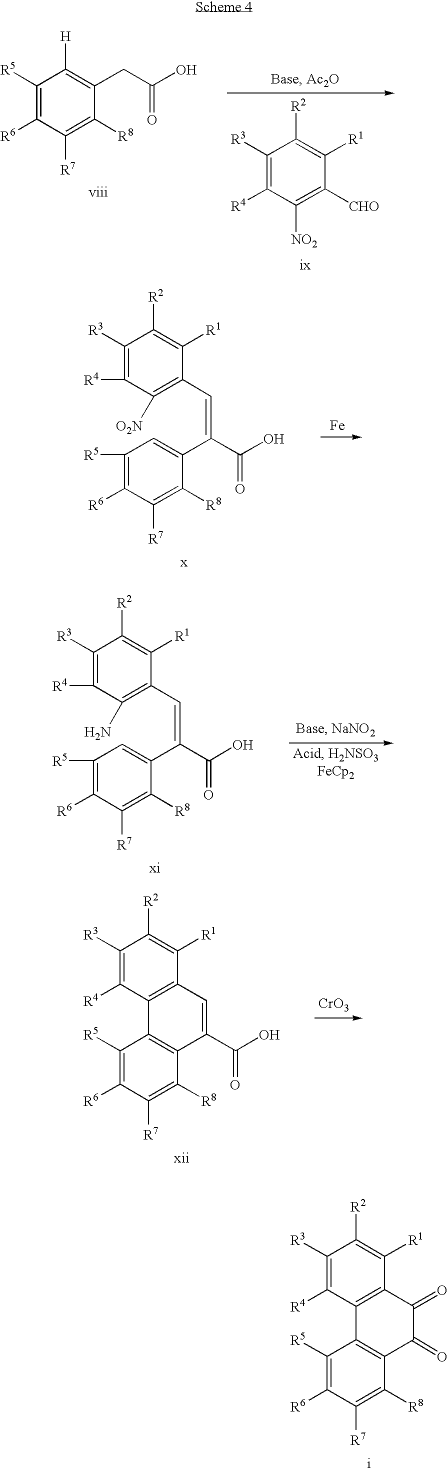

Scheme 5 describes an alternate route for the synthesis for phenanthrenequinone i. Treatment of an appropriately substituted bromo-phenylacetic ester xiii (which could be prepared for example, by esterification of phenylacetic acids, displacement of activated aryl fluorides with malonate derivatives followed by decarboxylation, or Wolffe rearrangement of benzoic acids) with an aryl boronic acid xiv in the presence of a catalyst such as Pd(PPh₃)₄ and in the presence of a base, such as cesium fluoride, in an suitable solvent, such as DMF or DME, followed by hydrolysis of the ester with a base such as sodium hydroxide in a suitable mixture of solvents such as THF and methanol produces the phenylacetic acid xv. Conversion of the acid xv into its acyl chloride upon treatment with a suitable reagent such as thionyl chloride or oxalyl chloride in the presence of a catalytic amount of DMF in a solvent such as 1,2-dichloroethane or THF, followed by intramolecular cyclisation in the presence of a Lewis acid such as aluminum trichloride in a solvent such as 1,2-dichloroethane produces the phenanthrol xvia. This phenanthrol xvia can be directly oxidized to phenanthrenequinone i by treatment with a catalytic amount of N,N′-Bis(salicylidene)ethylenediaminocobalt (II) hydrate [Co(SALEN)₂] in a solvent such as DMF, in the presence of air. Optionally, phenanthrol xvia could be further elaborated to phenanthrol xvib by the appropriate interconversion of any of the functional groups R¹ to R⁸, for example oxidation of a sulfide to a sulfone with an oxidizing agent such as MCPBA in a suitable solvent such as methylene chloride. Phenanthrol xvib can then be oxidized to phenanthrenequinone i as described above

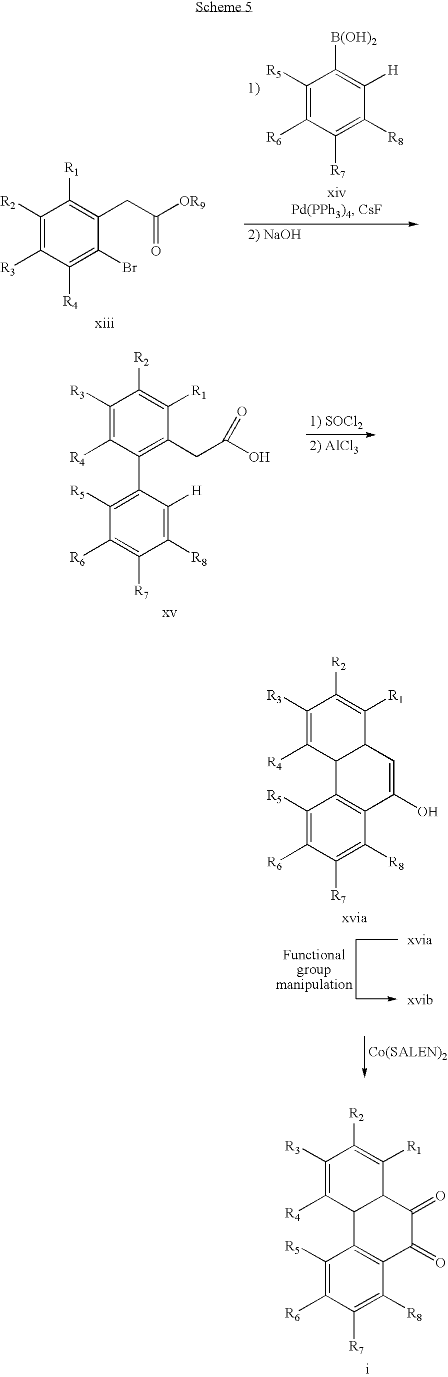

The imidazole secondary amine present in compounds of Formula I can be substituted as described in Scheme 6 by treating an appropriately functionalized phenanthrene imidazole I with a reagent such as an acylating agent or an alkylating agent such as methyl iodide in the presence of a base such as sodium hydride in a suitable solvent such as DMF, to produce the N-substituted imidazole xvii.

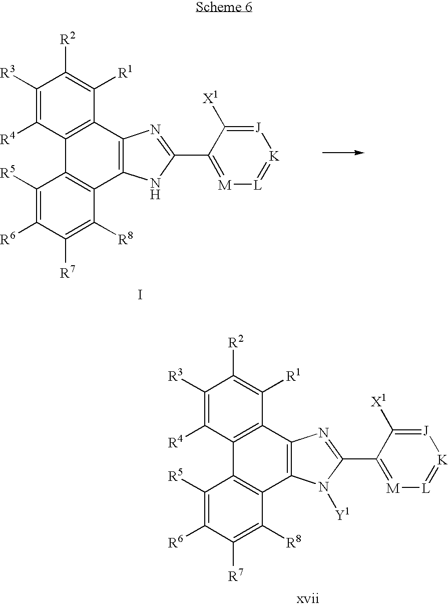

### EXAMPLES

The invention is exemplified by the following non-limiting examples:

### Example 12

### 2-(2-fluorophenyl)-1H-phenanthro[9,10-d]imidazole

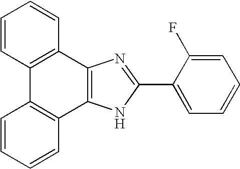

A mixture of phenanthrene-9,10-dione (980 mg, 4.71 mmol), ammonium hydrogencarbonate (1.5 g, 18.9 mmol) and 2-fluorobenzaldehyde (1 mL, 9.49 mmol) in acetic acid (30 mL) was heated at reflux for 19 hours. The mixture was then cooled to room temperature, poured into water and stirred for 2 hours. The mixture was filtered and the solids washed with water and hexanes. The solids were swished in a mixture of hexanes and diethyl ether overnight. After filtration, 2-(2-fluorophenyl)-1H-phenanthro[9,10-d]imidazole (1.12 g, 76%) was obtained as a brown solid.

### Example 18

### 6,9-dibromo-2-(2-chloro-6-fluorophenyl)-1H-phenanthro[9,10-d]imidazole

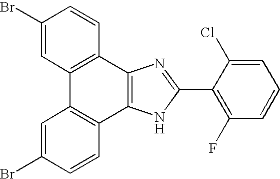

To a solution of 3,6-dibromophenanthrene-9,10-dione (15 g, 41 mmol, Bhatt, Tetrahedron, 1963, 20, 803) in acetic acid (500 mL) was added NH₄HCO₃ (13 g, 164 mmol) followed by 2-fluoro-6-chlorobenzaldehyde (13 g, 82 mmol). The solution was stirred overnight at 130° C., then cooled to room temperature and poured into water (1.5 L). The mixture was filtered, the solids obtained were washed with water and then swished in diethyl ether to afford 6,9-dibromo-2-(2-chloro-6-fluorophenyl)-1H-phenanthro[9,10-d]imidazole (18 g, 87%) as a beige solid. ¹H NMR δ (ppm) (400 MHz, Acetone-d₆): 12.85 (1H, bs), 9.19-9.0 (2H, m), 8.68-8.50 (1H, m), 8.45-8.30 (1H, m), 7.90 (2H, d), 7.71-7.61 (1H, m), 7.52 (1H, d), 7.40 (1H, t).

### Example 23

### Dimethyl 2-(2-chloro-6-fluorophenyl)-1H-phenanthro[9,10-d]imidazole-6,9-dicarboxylate

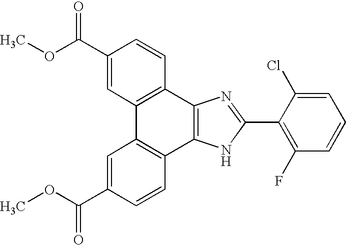

To a solution of 6,9-dibromo-2-(2-chloro-6-fluorophenyl)-1H-phenanthro[9,10-d]imidazole (500 mg, 0.99 mmol) from Example 18, in DMF (2 mL) and methanol (2 mL) was added triethyl amine (345 μL, 2.48 mmol), Pd(OAc)₂ (7 mg, 0.029 mmol) and dppf (33 mg, 0.06 mmol). The mixture was purged under vacuum and backfilled with carbon monoxide (3 times), then heated at 60° C. under an atmosphere of carbon monoxide overnight. The reaction mixture was cooled to room temperature, diluted with ethyl acetate and water. The aqueous layer was extracted with ethyl acetate, the organic layer washed with successively with water and brine, dried over Na₂SO₄, filtered and concentrated. The crude material was purified by flash chromatography to afford dimethyl 2-(2-chloro-6-fluorophenyl)-1H-phenanthro[9,10-d]imidazole-6,9-dicarboxylate. ¹H NMR 6 (ppm) (400 MHz, DMSO-d₆): 14.2 (1H, bs), 9.4 (2H, bs), 8.70-8.46 (2H, m), 8.34-8.27 (2H, m), 7.78-7.70 (1H, m), 7.65 (1H, d), 7.57 (1H, t), 4.02 (6H, s).

### Example 41

### 2-[6-bromo-2-(2-chloro-6-fluorophenyl)-1H-phenanthro[9,10-d]imidazol-9-yl]-1,1,1,3,3,3-hexafluoropropan-2-ol

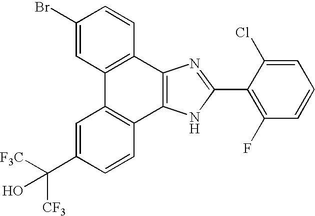

To a solution of 6,9-dibromo-2-(2-chloro-6-fluorophenyl)-1H-phenanthro[9,10-d]imidazole (10 g, 20 mmol) from Example 18, in THF at −78° C. was added methyllithium (1.6 M in Et₂O, 12.5 mL), followed by butyllithium (2.5 M in hexanes, 8 mL). Hexafluoroacetone was bubbled into the resulting yellow-green suspension until the mixture became homogeneous. The reaction mixture was removed from the cooling bath for 15 minutes, and then re-cooled to −78° C. and then quenched with saturated ammonium acetate. The mixture was warmed to room temperature and diluted with ethyl acetate. The aqueous layer was extracted with ethyl acetate, the organic layer washed with brine, dried over Na₂SO₄, filtered and concentrated. The crude material was recrystallized from toluene to afford 2-[6-bromo-2-(2-chloro-6-fluorophenyl)-1H-phenanthro[9,10-d]imidazol-9-yl]-1,1,1,3,3,3-hexafluoropropan-2-ol (8.5 g, 72%). ¹H NMR δ (ppm) (400 MHz, DMSO-d₆, mixture of tautomers): 14.1 (0.5 Ha, bs), 14.05 (0.5 Hb, bs), 9.15-8.90 (3Ha,b, td), 8.68 (0.5 Ha, d), 8.51 (1 Ha,b, t), 8.39 (0.5 Hb, d), 8.08-7.90 (2 Ha,b, m), 7.75-7.65 (1 Ha,b, m), 7.62 (1 Ha,b, dd), 7.52 (1 Ha,b, t).

### Example 62

### 2-(5-azido-2-iodophenyl)-1H-phenanthro[9,10-d]imidazole

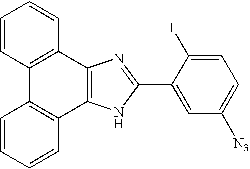

### Step 1: 2-(2-iodo-5-nitrophenyl)-1H-phenanthro[9,10-d]imidazole

A mixture of phenanthrene-9,10-dione (148 mg, 0.71 mmol), ammonium acetate (1.1 g) and 2-iodo-5-nitrobenzaldehyde (270 mg, 1 mmol) in acetic acid (8 mL) was heated at reflux for 1 hour. The mixture was then cooled to room temperature, poured into water and stirred for 10 minutes. The mixture was filtered and the solids obtained were washed with water and hexanes. The solids were dried under high vacuum to afford 2-(2-iodo-5-nitrophenyl)-1H-phenanthro[9,10-d]imidazole (250 mg) as a yellow solid.

### Step 2: 4-iodo-3-(1H-phenanthro[9,10-d]imidazol-2-yl)aniline

A mixture of 2-(2-iodo-5-nitrophenyl)-1H-phenanthro[9,10-d]imidazole (91 mg) from Step 1 above, nickel boride (60 mg, TL, 1993, 34, 3083) and HCl (1 N, 1 mL) in MeOH (4 mL) was stirred at 60° C. for 2 hours. The reaction mixture was then poured into water. Concentrated ammonium hydroxide was added until the mixture became basic. The aqueous layer was extracted with ethyl acetate, the organic layer washed with brine, dried over MgSO₄, filtered and concentrated. The crude material was purified by flash chromatography on silica to yield 4-iodo-3-(1H-phenanthro[9,10-d]imidazol-2-yl)aniline (60 mg).

### Step 3: 2-(5-azido-2-iodophenyl)-1H-phenanthro[9,10-d]imidazole

To a solution of 4-iodo-3-(1H-phenanthro[9,10-d]imidazol-2-yl)aniline (60 mg) from Step 2 above in acetic acid (3 mL) at 0° C. was added water (10 mL), followed by a solution of sodium nitrite (20 mg) in water. The reaction mixture was stirred at 0° C. for 30 minutes, after which a solution of sodium azide (20 mg) in water was added. After 30 minutes, the reaction mixture was diluted with water and the aqueous layer extracted with ethyl acetate. The organic layer was washed successively with water, sodium hydroxide (1 N) and brine, dried over MgSO₄, filtered and concentrated. The solid was dried under high vacuum to afford 2-(5-azido-2-iodophenyl)-1H-phenanthro[9,10-d]imidazole as a yellow solid. ¹H NMR δ (ppm) (400 MHz, Acetone-d₆): 12.55 (1H, bs), 8.91-8.80 (2H, m), 8.72-8.62 (1H, m), 8.46-8.35 (1H, m), 8.10 (1H, d), 7.75-7.60 (4H, m), 7.53 (1H, d), 7.05 (1H, dd).

### Example 63

### 9-bromo-2-(2-chloro-6-fluorophenyl)-6-(methylsulfonyl)-1H-phenanthro[9,10-d]imidazole

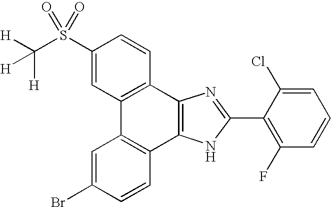

### Step 1: 1-bromo-4-{2-[4-(methylthio)phenyl]vinyl} benzene

KHMDS (0.5 M in toluene, 6.4 mL) was added to a −78° C. suspension of (4-bromobenzyl)triphenylphosphonium bromide (1.64 g, 3.2 mmol) in THF (10 mL). The resulting orange suspension was stirred at −78° C. for 0.5 hour, after which 4-(methylthio)benzaldehyde (430 μL, 3.2 mmol) was added. The reaction mixture was warmed to 0° C. and stirred for 1.5 hours at this temperature, after which it was quenched with water. The aqueous layer was extracted with ethyl acetate, the organic layer washed with brine, dried over Na₂SO₄, filtered and concentrated. The crude material was purified by flash chromatography on silica (0-2% ethyl acetate in hexanes) to yield 1-bromo-4-{2-[4-(methylthio)phenyl]vinyl}benzene (424 mg, 43%) as a mixture of isomers.

### Step 2: 3-bromo-6-(methylthio)phenanthrene

To a 250 mL vessel equipped with a pyrex inner water-cooled jacket was added 1-bromo-4-{2-[4-(methylthio)phenyl]vinyl}benzene (423 mg, 1.39 mmol) from Step 1 above, followed by a solution of iodine (530 mg, 2.09 mmol) in cyclohexane (250 mL) and THF (3.5 mL). The stirred solution was first degassed by bubbling nitrogen and was then exposed to UV light for 30 hrs by inserting a 450 W medium pressure mercury lamp in the inner insert. The reaction was quenched with 10% Na₂S₂O₃ and the aqueous layer was extracted with ethyl acetate. The combined organic layers were washed with brine, dried over Na₂SO₄, filtered and the volatiles removed under reduced pressure. The crude material was purified by flash chromatography on silica (100% hexanes) to yield 3-bromo-6-(methylthio)phenanthrene (260 mg) as a yellow solid.

### Step 3: 3-bromo-6-(methylsulfonyl)phenanthrene

MCPBA (approx. 77%, 450 mg) was added to a room temperature solution of 3-bromo-6-(methylthio)phenanthrene (260 mg, 0.86 mmol) from Step 2 above, in CH₂Cl₂. The reaction mixture was stirred at room temperature for 3 hours, then quenched with saturated sodium bicarbonate. The aqueous layer was extracted with CH₂Cl₂. The combined organic layers were washed successively with 10% Na₂S₂O₃; and brine, then dried over Na₂SO₄, filtered and concentrated. The crude material was purified by flash chromatography on silica (20-80% ethyl acetate in hexanes) to yield 3-bromo-6-(methylsulfonyl)phenanthrene (276 mg) as a white solid.

### Step 4: 3-bromo-6-(methylsulfonyl)phenanthrene-9,10-dione

To a solution of 3-bromo-6-(methylsulfonyl)phenanthrene (276 mg, 0.82 mmol) from Step 3 above in acetic acid (15 mL) was added CrO₃ (330 mg, 3.3 mmol). The reaction mixture was stirred for 1 hour at 120° C., then cooled to room temperature and poured into water. The resulting suspension was stirred for 0.5 hour, filtered and the solids obtained were washed with water. The solids were dried under high vacuum to afford 3-bromo-6-(methylsulfonyl)phenanthrene-9,10-dione (170 mg, 57%) as a yellow solid.

### Step 5: 9-bromo-2-(2-chloro-6-fluorophenyl)-6-(methylsulfonyl)-1H-phenanthro[9,10-d]imidazole

This imidazole was prepared as described in Example 18, substituting 3-bromo-6-(methylsulfonyl)phenanthrene-9,10-dione from Step 4 above for 3,6-dibromophenanthrene-9,10-dione, to afford 9-bromo-2-(2-chloro-6-fluorophenyl)-6-(methylsulfonyl)-1H-phenanthro[9,10-d]imidazole as a beige solid. ¹H NMR δ (ppm) (400 MHz, DMSO-d₆, mixture of tautomers): 14.22 (1 Ha, s), 14.12 (1 Hb, s), 9.45 (1 Ha,b, d), 9.27 (1 Ha,b, dd).

### Example 69

### 2-[6-bromo-2-(2-chloro-6-fluorophenyl)-1H-phenanthro[9,10-d]imidazol-9-yl]-1,1,1-trifluoropropan-2-ol

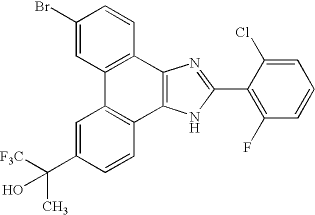

Methyllithium (1.6 M in Et₂O, 5 mL) was added to a −78° C. solution of 6,9-dibromo-2-(2-chloro-6-fluorophenyl)-1H-phenanthro[9,10-d]imidazole (3.91 g, 7.75 mmol) from Example 18 in THF (130 mL), followed by the addition of butyllithium (2.5 M in hexanes, 3.1 mL). The resulting green suspension was stirred for 10 minutes, after which ethyl trifluoroacetate (4 mL, 33.6 mmol) was added. The reaction mixture was stirred at −78° C. for 10 minutes, then warmed to 0° C. and stirred for 0.5 hour. The reaction was quenched with 25% ammonium acetate. The aqueous layer was extracted with ethyl acetate, the organic layer washed with brine, dried over Na₂SO₄, filtered and concentrated. To a solution of this crude product in THF (100 mL) at −78° C. was added methylmagnesium bromide (3 M in Et₂O, 8 mL). The reaction mixture was stirred at −78° C. for 15 minutes, then warmed to 0° C. and stirred for 0.5 hour. The reaction was quenched with 25% ammonium acetate. The aqueous layer was extracted with ethyl acetate, the organic layer was washed with brine, dried over Na₂SO₄, filtered and concentrated. The crude material was purified by flash chromatography on silica (20-60% ethyl acetate in hexanes) to yield 2-[6-bromo-2-(2-chloro-6-fluorophenyl)-1H-phenanthro[9,10-d]imidazol-9-yl]-1,1,1-trifluoropropan-2-ol (2.2 g, 53%) as a yellow foam. ¹H NMR δ (ppm) (400 MHz, Acetone-d₆): 13.9 (1H, s), 9.20-9.03 (2H, m), 8.55-8.33 (2H, m), 8.03-7.92 (2H, m), 7.74-7.71 (1H, m), 7.69-7.62 (1H, m), 7.54 (1H, t), 6.89 (1H, bs), 1.94 (3H, s).

### Example 86

### 2-[2-(2-chloro-6-fluorophenyl)-6-(cyclopropylethynyl)-1H-phenanthro[9,10-d]imidazol-9-yl]-1,1,1,3,3,3-hexafluoropropan-2-ol

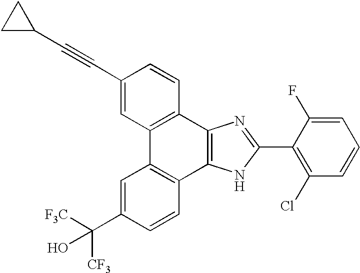

To a solution of 2-[6-bromo-2-(2-chloro-6-fluorophenyl)-1H-phenanthro[9,10-d]imidazol-9-yl]-1,1,1,3,3,3-hexafluoropropan-2-ol (400 mg) from Example 41, in DMF (20 mL) was added ethynylcyclopropane (357 μL), Pd(PPh₃)₄ (78 mg) and triethylamine (3 mL). The reaction mixture was stirred at room temperature for 5 minutes, after which CuI (19 mg) was added, and the reaction mixture heated at 65° C. for 4 hours. The reaction was quenched with ethyl acetate, water and concentrated ammonium hydroxide. The aqueous layer was extracted with ethyl acetate, the organic layer washed with brine, dried over Na₂SO₄, filtered and concentrated. The crude material was purified by flash chromatography on silica (25% ethyl acetate in hexanes) to yield 2-[2-(2-chloro-6-fluorophenyl)-6-(cyclopropylethynyl)-1H-phenanthro[9,10-d]imidazol-9-yl]-1,1,1-trifluoropropan-2-ol (320 mg, 82%).). ¹H NMR δ (ppm) (400 MHz, Acetone-d₆, mixture of tautomers): 9.42-9.25 (1 Ha,b, m), 8.95-8.80 (1.5 Ha,b, m), 8.72-8.54 (1 Ha,b, m), 8.48-8.38 (0.5 Ha,b, m), 8.20-8.07 (1 Ha,b, m), 8.05-7.89 (1 Ha,b, m), 7.80-7.70 (1 Ha,b, m), 7.62-7.55 (1 Ha,b, m), 7.45 (1 Ha,b, d), 7.30 (1 Ha,b, t), 1.68-1.50 (1 Ha,b, m), 1.00-0.89 (2 Ha,b, m), 0.86-0.78 (2 Ha,b, m).

### Example 90

### 2-[2-(2-chloro-6-fluorophenyl)-6-pyridin-3-yl-1H-phenanthro[9,10-d]imidazol-9-yl]-1,1,3,3,3-hexafluoropropan-2-ol

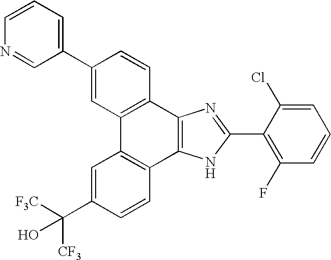

To a solution of 2-[6-bromo-2-(2-chloro-6-fluorophenyl)-1H-phenanthro[9,10-d]imidazol-9-yl]-1,1,1,3,3,3-hexafluoropropan-2-ol (650 mg, 1.09 mmol) from Example 41 in n-propanol (10 mL) was added 3-(1,3,2-dioxaborinan-2-yl)pyridine (180 mg, 1.1 mmol), triphenyl phosphine (50 mg, 0.21 mmol) and sodium carbonate (2 M, 1.7 mL). The mixture was degassed 3 times and backfilled with nitrogen. Pd(OAc)₂ (15 mg, 0.07 mmol) was then added and the reaction mixture heated at 90° C. under nitrogen atmosphere for 3 hours. The reaction was quenched with 25% ammonium acetate and ethyl acetate. The aqueous layer was extracted with ethyl acetate, the organic layer washed with brine, dried over Na₂SO₄, filtered and concentrated. The crude material was purified by flash chromatography on silica (20-100% ethyl acetate in hexanes) to yield 2-[2-(2-chloro-6-fluorophenyl)-6-pyridin-3-yl-1H-phenanthro[9,10-d]imidazol-9-yl]-1,1,1,3,3,3-hexafluoropropan-2-ol (400 mg, 62%) as a beige solid.

### Example 91

### 1-[6-bromo-2-(2-chloro-6-fluorophenyl)-1H-phenanthro[9,10-d]imidazol-9-yl]-2,2,2-trifluoroethane-1,1-diol

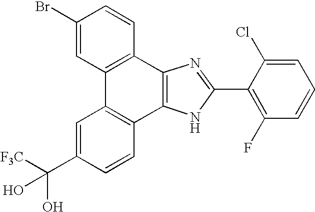

To a solution of 6,9-dibromo-2-(2-chloro-6-fluorophenyl)-1H-phenanthro[9,10-d]imidazole (500 mg, 1 mmol) from Example 18, in THF (13 mL) at −78° C. was added methyllithium (1.6 M in Et₂O, 625 μL), followed by butyllithium (2.5 M in hexanes, 400 μL). Ethyl trifluoroacetate (596 μL) was then added in one portion and the reaction mixture warmed up to room temperature. It was then re-cooled to −78° C. and quenched with 25% ammonium acetate. The aqueous layer was extracted with ethyl acetate, the organic layer washed with brine, dried over Na₂SO₄, filtered and concentrated. The crude material was purified by flash chromatography on silica (20-70% ethyl acetate in hexanes) to afford 1-[6-bromo-2-(2-chloro-6-fluorophenyl)-1H-phenanthro[9,10-d]imidazol-9-yl]-2,2,2-trifluoroethane-1,1-diol (184 mg, 34%). ¹H NMR δ (ppm) (400 MHz, DMSO-d₆, mixture of tautomers): 14.25 (0.5 Ha, bs), 14.18 (0.5 Hb, bs), 9.50-9.40 (1Ha,b, m), 9.10 (1 Ha,b, d), 8.72 (0.5 Ha, d), 8.58-8.48 (1 Ha,b, dd), 8.38-8.28 (1.5 Ha,b, m), 8.06-7.91 (1Ha,b, dd), 7.78-7.70 (1 Ha,b, m), 7.62 (1 Ha,b, d), 7.52 (1 Ha,b, t).

### Example 99

### 2-[2-(2-chloro-6-fluorophenyl)-6-iodo-1H-phenanthro[9,10-d]imidazol-9-yl]-1,1,1,3,3,3-hexafluoropropan-2-ol

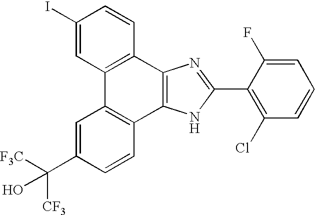

To a solution of 2-[6-bromo-2-(2-chloro-6-fluorophenyl)-1H-phenanthro[9,10-d]imidazol-9-yl]-1,1,1,3,3,3-hexafluoropropan-2-ol (400 mg) from Example 41, in DMF (4 mL) was added bis(tributyltin) (1.4 mL) followed by toluene (2 mL) for complete dissolution. Pd₂ dba₃ (31 mg) and triphenylarsine (41 mg) were then added and the reaction mixture heated at 80° C. for 5 hours. Pd(PPh₃)₄ was then added and the reaction mixture heated at 80° C. for 3 hours. The material was purified by flash chromatography on silica (25% ethyl acetate in hexanes). To this product (107 mg) in CHCl₂, was added iodine. The reaction mixture was stirred at room temperature for 1 hour, after which it was quenched with 10% Na₂S₂O₃. The aqueous layer was extracted with ethyl acetate, the organic layer washed with brine, dried over Na₂SO₄, filtered and concentrated. The crude material was purified by flash chromatography on silica (25% ethyl acetate in hexanes) to afford 2-[2-(2-chloro-6-fluorophenyl)-6-iodo-1H-phenanthro[9,10-d]imidazol-9-yl]-1,1,1-trifluoropropan-2-ol (70 mg). ¹H NMR δ (ppm) (400 MHz, Acetone-d₆, mixture of tautomers): 9.26 (2 Ha,b, td), 8.82 (0.5 Ha, d), 8.53 (1 Ha,b, dd), 8.26 (0.5 Hb, d), 8.14-8.09 (2 Ha,b, m), 7.84 (1 Ha,b, d), 7.70-7.65 (1 Ha,b, m), 7.54 (1 Ha,b, d), 7.40 (1 Ha,b, t).

### Example 101

### 2-[2-(2-chloro-6-fluorophenyl)-6-(phenylthio)-1H-phenanthro[9,10-d]imidazol-9-yl]-1,1,1,3,3,3-hexafluoropropan-2-ol

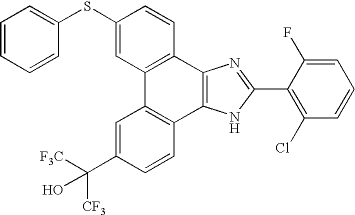

To a solution of 2-[2-(2-chloro-6-fluorophenyl)-6-iodo-1H-phenanthro[9,10-d]imidazo-9-yl]-1,1,1,3,3,3-hexafluoropropan-2-ol (56 mg) from Example 99 in NMP (3 mL) was added Pd₂ dba₃ (6 mg), dpppf (16 mg) and triethyl amine (24 μL). The mixture was stirred for 5 minutes, after which benzenethiol (18 μL) was added and the mixture heated at 60° C. for 2 hours, then at 75° C. for 1 hour. Pd(PPh₃)₄ was then added and the reaction mixture was stirred at 75° C. for 6 hours. It was quenched with water and ethyl acetate. The aqueous layer was extracted with ethyl acetate, the organic layer washed with brine, dried over Na₂SO₄, filtered and concentrated. The crude material was purified by flash chromatography on silica (25% ethyl acetate in hexanes) to afford 2-[2-(2-chloro-6-fluorophenyl)-6-(phenylthio)-1H-phenanthro[9,10-d]imidazol-9-yl]-1,1,1,3,3,3-hexafluoropropan-2-ol. ¹H NMR δ (ppm) (400 MHz, Acetone-d₆): 9.20 (1H, d), 8.85 (1H, s), 8.76 (1H, dd), 8.50 (1H, dd), 8.13-8.08 (1H, m), 7.83-7.65 (3H, m), 7.55 (1H, d), 7.50-7.32 (6H, m).

### Example 109

### 2-[6-(benzylsulfonyl)-2-(2-chloro-6-fluorophenyl)-1H-phenanthro[9,10-d]imidazol-9-yl]-1,1,1,3,3,3-hexafluoropropan-2-ol

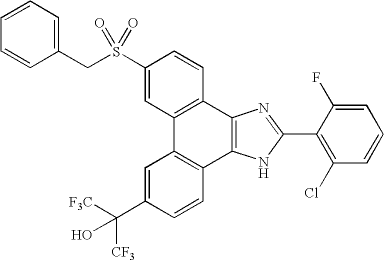

To a solution of 2-[2-(2-chloro-6-fluorophenyl)-6-iodo-1H-phenanthro[9,10-d]imidazol-9-yl]-1,1,1,3,3,3-hexafluoropropan-2-ol (53 mg) from Example 99 in NMP (3 mL) was added triethyl amine (23 μL) and Pd(PPh₃)₄ (10 mg). The reaction mixture was stirred at room temperature for 5 minutes, after which phenylmethanethiol (19 μL) was added and the reaction mixture heated at 80° C. for 4 hours. Pd₂ dba₃/dpppf (0.2 equivalents) were then added and the reaction mixture heated at 80° C. for 2 hours. It was quenched with water and ethyl acetate. The aqueous layer was extracted with ethyl acetate, the organic layer washed with brine, dried over Na₂SO₄, filtered and concentrated. The crude material was purified by flash chromatography on silica (25% ethyl acetate in hexanes). To a solution of this product in methanol was added Oxone (102 mg) and the reaction mixture was stirred at room temperature for 3 hours. After usual workup as described above, the crude material was purified by flash chromatography on silica (25% ethyl acetate in hexanes) to afford 2-[6-(benzylsulfonyl)-2-(2-chloro-6-fluorophenyl)-1H-phenanthro[9,10-d]imidazol-9-yl]-1,1,1,3,3,3-hexafluoropropan-2-ol (19 mg). ¹H NMR δ (ppm) (400 MHz, Acetone-d₆): 9.14 (1H, d), 9.01 (1H, d), 8.85 (1H, t), 8.60 (1H, t), 8.18-8.05 (1H, m), 8.03-7.92 (2H, m), 7.74-7.68 (1H, m), 7.57 (1H, d), 7.44 (1H, t), 7.26 (5H, s), 4.70 (2H, s).

### Example 114

### N-[2-(2-chloro-6-fluorophenyl)-1H-phenanthro[9,10-d]imidazol-6-yl]acetamide

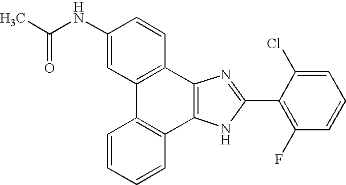

### Step 1: 1-1-(3-phenanthryl)ethanone oxime

To a solution of 3-acetylphenanthrene (29.14 g, 132 mmol) in absolute ethanol (110 mL) was added hydroxylamine hydrochloride (23 g, 331 mmol) and pyridine (40 mL). The mixture was heated at reflux for 4 hours, then cooled to room temperature and concentrated under reduced pressure. The resulting solid was suspended in ice-water and filtered. The solid was washed with water and subsequently dried under vacuum to afford 1-1-(3-phenanthryl)ethanone oxime (45.5 g).

### Step 2: N-3-phenanthrylacetamide

To polyphosphoric acid (250 g) mechanically stirred at 100° C. was added 1-1-(3-phenanthryl)ethanone oxime (14.6 g) from Step 1 above, portionwise. The mixture was stirred at 100° C. under nitrogen atmosphere for 2.5 hours, then poured into a mixture of ice and water while stirring. The aqueous layer was extracted with chloroform. The combined organic layers were washed twice with 10% potassium carbonate, twice with water, dried over MgSO₄ and the volatiles were removed under reduced pressure. The residue was dried under high vacuum to afford N-3-phenanthrylacetamide (9.44 g) as a beige solid.

### Step 3: N-(9,10-dioxo-9,10-dihydrophenanthren-3-yl)acetamide

A mixture of N-3-phenanthrylacetamide (235 mg, 1 mmol) from Step 2 above, and chromium trioxide (400 mg, 4 mmol) in acetic acid (10 mL) was heated at reflux for 45 minutes. The mixture was cooled to room temperature, then poured into water. The aqueous layer was extracted with ethyl acetate. The combined organic layers were washed successively with water and brine, dried over MgSO₄ and concentrated under reduced pressure. The residue was triturated in chloroform and filtered. The solids obtained were washed with chloroform and dried under high vacuum to yield N-(9,10-dioxo-9,10-dihydrophenanthren-3-yl)acetamide (95 mg) as an orange solid.

### Step 4: N-[2-(2-chloro-6-fluorophenyl)-1H-phenanthro[9,10-d]imidazol-6-yl]acetamide

To a solution of N-(9,10-dioxo-9,10-dihydrophenanthren-3-yl)acetamide (95 mg, 0.36 mmol) from Step 3 above, in acetic acid (3 mL) was added 2-chloro-6-fluorobenzaldehyde (114 mg, 0.72 mmol) and ammonium acetate (552 mg, 7.2 mmol). The mixture was heated at reflux for 2 hours, then cooled to room temperature. Water was added to the reaction mixture and the resulting solid filtered, washed with water and hexanes and dried under high vacuum to afford N-[2-(2-chloro-6-fluorophenyl)-1H-phenanthro[9,10-d]imidazol-6-yl]acetamide (142 mg) as an off-white solid. ¹H NMR δ (ppm) (400 MHz, Acetone-d₆ with added TFA): 9.70 (2H, bs), 9.38 (1H, s), 8.78 (1H, m), 8.60 (1H, m), 8.52 (1H, d), 8.05 (1H, d), 7.90 (3H, m), 7.70 (1H, d), 7.60 (1H, t), 2.25 (3H, s).

### Example 118

### 2-(2-chloro-6-fluorophenyl)-6-(morpholin-4-ylsulfonyl)-1H-phenanthro[9,10-d]imidazole

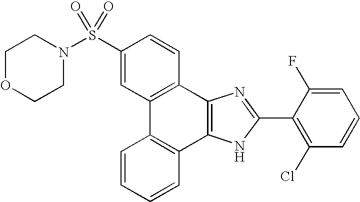

### Step 1: 1-bromo-4-[2-phenylvinyl]benzene

To a solution of benzyltriphenylphosphonium chloride (5.23 g, 13.4 mmol) in DMF (20 mL) at 0° C., was added NaH (60% dispersion in oil, 620 mg) portionwise. The mixture was stirred at 0° C. for 20 minutes, after which 4-bromobenzaldehyde (2.5 g, 13.5 mmol) was added. The reaction mixture was stirred at 0° C. for 15 minutes, then warmed to room temperature and stirred for 2 hours. The reaction mixture was quenched with 25% ammonium acetate. The aqueous layer was extracted with ethyl acetate, the organic layer washed with brine, dried over Na₂SO₄, filtered and concentrated. The crude material was dissolved in hot ethanol and left at room temperature. The resulting solids were filtered and the mother liquour was concentrated under reduced pressure. The residue was dissolved in hot cyclohexanes and then cooled to room temperature. The solids obtained were filtered and the mother liquour was concentrated. The residue was purified by flash chromatography on silica (100% hexanes) to afford 1-bromo-4-[2-phenylvinyl]benzene (1.77 g, 50%) as a mixture of isomers.

### Step 2: 3-bromophenanthrene

To a 250 mL vessel equipped with a pyrex inner water-cooled jacket was added 1-bromo-4-[2-phenylvinyl]benzene (520 mg, 2.0 mmol) from Step 1 above, in cyclohexane (230 mL) and THF (5 mL). To this solution was added iodine (770 mg, 3.0 mmol). The stirred solution was first degassed by bubbling nitrogen and was then exposed to UV light for 16 hours by inserting a 450 W medium pressure mercury lamp in the inner insert. The reaction was quenched with 10% Na₂S₂O₃ and the aqueous layer was extracted with ethyl acetate. The combined organic layers were washed with brine, dried over Na₂SO₄ and the volatiles were removed under reduced pressure. The crude material was purified by flash chromatography on silica (100% hexanes) to afford 3-bromophenanthrene (486 mg, 94%) as a white solid.

### Step 3: 3-bromophenanthrene-9,10-dione

To a solution of 3-bromophenanthrene (486 mg, 1.29 mmol) from Step 2 above, in acetic acid (30 mL) was added CrO₃ (800 mg, 8.0 mmol). The reaction was stirred at reflux for 1 hour, then cooled to room temperature and poured into water. The resulting suspension was stirred for 0.5 hour and filtered. The solids obtained were washed with water and hexanes. The solids were then swished in hexanes and filtered to afford 3-bromophenanthrene-9,10-dione (381 mg, 70%) as a yellow solid.

This quinone could also be prepared by the following procedure:

To a solution of phenanthrene-9,10-dione (10 g, 48 mmol) in nitrobenzene (50 mL) was added benzoyl peroxide (480 mg, 2 mmol) and bromine (2.34 mL, 45 mmol). The reaction mixture was heated at 80° C., while being irradiated with a sunlamp, for 45 minutes. The reaction mixture was cooled to room temperature. Ethyl acetate (150 mL) was added and the mixture triturated for 5 minutes. The resulting solids were filtered, washed with ethyl acetate and dried under high vacuum to afford 3-bromophenanthrene-9,10-dione (5.53 g) as a yellow solid.

### Step 4: 6-bromo-2-(2-chloro-6-fluorophenyl)-1H-phenanthro[9,10-d]imidazole

This imidazole was prepared as described in Example 18, substituting 3-bromophenanthrene-9,10-dione from Step 3 above for 3,6-dibromophenanthrene-9,10-dione to afford 6-bromo-2-(2-chloro-6-fluorophenyl)-1H-phenanthro[9,10-d]imidazole.

### Step 5: 2-(2-chloro-6-fluorophenyl)-6-(morpholin-4-ylsulfonyl)-1H-phenanthro[9,10-d]imidazole

To a solution of 6-bromo-2-(2-chloro-6-fluorophenyl)-1H-phenanthro[9,10-d]imidazole (125 mg) from Step 4 above, in THF at −78° C. was added methyllithium (1.6 M in Et₂O, 183 μL), followed by butyllithium (1.6 M in hexanes, 183 μL). The reaction mixture was stirred for 30 minutes, after which another equivalent of butyllithium was added and the reaction mixture was stirred for a further 30 minutes. Sulfur dioxide gas was bubbled through the reaction mixture, which was stirred at −78° C. for 10 minutes and then warmed to room temperature and stirred for 1 hour. The reaction mixture was concentrated under reduced pressure. The residue was taken up in ethyl acetate and phosphate buffer. To this was added N-chlorosuccinimide (118 mg) and the mixture stirred at room temperature for 1 hour. It was quenched with water and ethyl acetate. The aqueous layer was extracted with ethyl acetate. The combined organic layers were washed with brine, dried over Na₂SO₄ and the volatiles were removed under reduced pressure. To the crude material dissolved in THF, was added morpholine and the reaction mixture stirred overnight at room temperature. After workup as described above, the crude material was purified by flash chromatography on silica (25-50% ethyl acetate in hexanes) to afford 2-(2-chloro-6-fluorophenyl)-6-(morpholin-4-ylsulfonyl)-1H-phenanthro[9,10-d]imidazole. ¹H NMR δ (ppm) (400 MHz, DMSO-d₆, mixture of tautomers): 9.14 (1 Ha,b, d), 8.97 (1 Ha,b, dd), 8.77 (0.6 Ha,b, d), 8.61 (0.8 Ha,b, dd), 8.44 (0.6 Ha,b, d), 8.06 (1 Ha,b, dd), 7.89-7.70 (3 Ha,b, m), 7.64 (1 Ha,b, d), 7.55 (1 Ha,b, t), 3.66 (4 Ha,b, m), 3.01 (4 Ha,b, m).

### Example 137

### 9-bromo-6-chloro-2-(2-chloro-6-fluorophenyl)-1H-phenanthro[9,10-d]imidazole

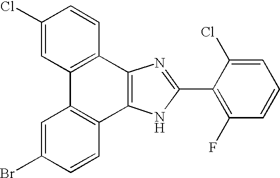

### Step 1: 1-bromo-4-[2-(4-chlorophenyl)vinyl]benzene

To a solution of (4-bromobenzyl)triphenylphosphonium bromide (396 g; 0.77 mol) in 2.5 L of DMF at 0° C., was added 37 g (0.92 mol) of NaH (60% in oil) in four portions. The solution was stirred for 1 hour at 0° C., followed by the addition of 109 g (0.77 mol) of 4-chlorobenzaldehyde in two portions. This mixture was warmed to room temperature, stirred for 1 hour and quenched by pouring the reaction mixture into a 5° C. mixture of 10 L of water and 2.5 L of Et₂O. The aqueous layer was extracted with Et₂O, the combined organic layers were washed with brine and dried over Na₂SO₄. The volatiles were removed under reduced pressure and the residue was dissolved in 1.5 L of cyclohexane and filtered through a pad of silica gel, which was subsequently washed with cyclohexane. 16 g of one isomer crystallized out of the solution as a white solid. After evaporation of the volatiles, 166 g of the other isomer of 1-bromo-4-[2-(4-chlorophenyl)vinyl]benzene was isolated.

### Step 2: 3-bromo-6-chlorophenanthrene

A 2 L vessel equipped with a pyrex inner water-cooled jacket was charged with 5.16 g (17 mmol) of 1-bromo-4-[2-(4-chlorophenyl)vinyl]benzene from Step 1 above, 2 L of cyclohexane, 25 mL of THF, 25 mL of propylene oxide and 6.7 g (26 mmol) of iodine. The stirred solution was first degassed by bubbling nitrogen through the reaction mixture and was then exposed to UV light for 24 hours by inserting a 450 W medium pressure mercury lamp in the inner insert. The reaction was quenched with 10% Na₂S₂O₃ and the aqueous layer was extracted with ethyl acetate. The combined organic layers were washed with brine, dried over Na₂SO₄ and the volatiles were removed under reduced pressure. The residue was swished in a minimal amount of ethyl acetate to afford approx. 5 g of 3-bromo-6-chlorophenanthrene as a solid.

### Step 3: 3-Bromo-6-chlorophenanthrene-9,10-dione

To a solution of 3-bromo-6-chlorophenanthrene (1.71 g; 5.86 mmol) from Step 2 above, in 35 mL of acetic acid was added 2.3 g (23.5 mmol) of CrO₃. The mixture was stirred for 2 hours at 100° C., cooled to room temperature, poured into 300 mL of water and stirred for 1 hour. The suspension was filtered, the solids obtained were washed with water and Et₂O and dried under high vacuum to afford 1.67 g of 3-bromo-6-chlorophenanthrene-9,10-dione as a solid.

### Step 4: 9-bromo-6-chloro-2-(2-chloro-6-fluorophenyl)-1H-phenanthro[9,10-d]imidazole

To a solution of 3-bromo-6-chlorophenanthrene-9,10-dione (540 mg, 1.68 mmol) from Step 3 above, in acetic acid (40 mL) was added ammonium hydrogen carbonate (540 mg, 6.83 mmol) and 2-fluoro-6-chlorobenzaldehyde (540 mg, 3.41 mmol). The mixture was heated at reflux for 12 hours, then left at room temperature for 7 hours. The resulting white precipitate was filtered and the solids rinsed with water and hexanes. A solution of the solids in toluene was heated at reflux with a Dean Stark apparatus. The crystallized product was washed with toluene and hexanes and dried under high vacuum to afford 9-bromo-6-chloro-2-(2-chloro-6-fluorophenyl)-1H-phenanthro[9,10-d]imidazole (508 mg) as a white solid.

### Example 161

### 9-chloro-2-(2-chloro-6-fluorophenyl)-6-(1H-tetrazol-5-yl)-1H-phenanthro[9,10-d]imidazole

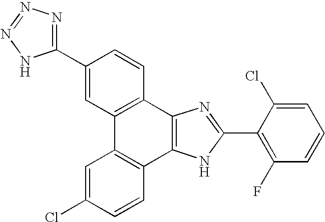

### Step 1: 9-chloro-2-(2-chloro-6-fluorophenyl)-1H-phenanthro[9,10-d]imidazole-6-carbonitrile

To a solution of 9-bromo-6-chloro-2-(2-chloro-6-fluorophenyl)-1H-phenanthro[9,10-d]imidazole (253 mg) from Example 137, in DMF (8 mL) was added zinc cyanide (129 mg) and Pd(PPh₃)₄ (64 mg). The reaction mixture was heated at 85° C. overnight, then quenched with water and ethyl acetate. The aqueous layer was extracted with ethyl acetate, the organic layer washed with brine, dried over Na₂SO₄, filtered and concentrated. The crude material was purified by flash chromatography on silica (50-75% ethyl acetate in hexanes) to yield 9-chloro-2-(2-chloro-6-fluorophenyl)-1H-phenanthro[9,10-d]imidazole-6-carbonitrile (36 mg).

### Step 2: 9-chloro-2-(2-chloro-6-fluorophenyl)-6-(1H-tetrazol-5-yl)-1H-phenanthro[9,10-d]imidazole

To a solution of 9-chloro-2-(2-chloro-6-fluorophenyl)-1H-phenanthro[9,10-d]imidazole-6-carbonitrile (36 mg) from Step 1 above, in DMF (3 mL) was added ammonium chloride (9 mg) and sodium azide (12 mg). The reaction mixture was heated at 100° C. overnight, then quenched with sodium bicarbonate and ethyl acetate. The aqueous layer was extracted with ethyl acetate. The organic layer was washed with brine, dried over Na₂SO₄, filtered and concentrated. The crude material was purified by flash chromatography on silica (10% methanol in methylene chloride) to yield 9-chloro-2-(2-chloro-6-fluorophenyl)-6-(1H-tetrazol-5-yl)-1H-phenanthro[9,10-d]imidazole (22 mg). ¹H NMR δ (ppm) (400 MHz, CD₃OD): 9.56 (1H, s), 8.99 (1H, s), 8.51-8.42 (3H, m), 7.74-7.63 (2H, m), 7.55 (1H, d), 7.39 (1H, t).

### Example 168

### 1-[6-chloro-2-(2-chloro-6-fluorophenyl)-1H-phenanthro[9,10-d]imidazol-9-yl]ethanone

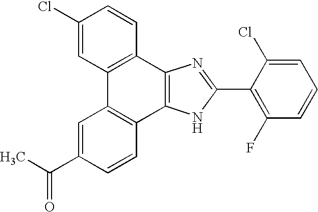

A round bottomed flask charged with 9-bromo-6-chloro-2-(2-chloro-6-fluorophenyl)-1H-phenanthro[9,10-d]imidazole (550 mg, 1.19 mmol) from Example 137, and PdCl₂(PPh₃)₂ (100 mg, 0.14 mmol) was purged with nitrogen for 15 minutes. DMF (10 mL) and tributyl-(1-ethoxy)-vinyltin (520 μL, 1.54 mmol) were added and the reaction mixture heated at 100° C. under nitrogen atmosphere for 2.5 hours. The reaction mixture was cooled to room temperature, quenched with 1 N HCl and stirred for 0.5 hours, after which it was neutralized with 25% NH₄OAc. The aqueous layer was extracted with THF, the organic layer was washed successively with water and brine, dried over Na₂SO₄, filtered and concentrated. The crude material was purified by flash chromatography on silica (10-35% ethyl acetate in hexanes) to afford 1-[6-chloro-2-(2-chloro-6-fluorophenyl)-1H-phenanthro[9,10-d]imidazol-9-yl]ethanone (250 mg, 50%) as a yellow solid. ¹H NMR δ (ppm) (400 MHz, DMSO-d₆): 14.1 (1H, two singlets), 9.46 (1H, d), 9.21 (1H, d), 8.59 (1H, dd), 8.43 (1H, dd), 8.28 (1H, dd), 7.88 (1H, dd), 7.81-7.72 (1H, m), 7.71-7.63 (1H, m), 7.55 (1H, t), 2.87 (1H, t).

### Example 182

### 6-chloro-2-(3,5-difluoropyridin-4-yl)-1H-phenanthro[9,10-d]imidazole

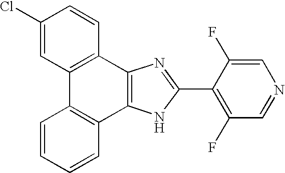

### Step 1: 1-(3-phenanthryl)ethanone oxime

A mixture of 1-(3-phenanthryl)ethanone (50 g, 0.23 mol) and hydroxylamine hydrochloride (40 g) in absolute ethanol (200 mL) was heated to reflux. Pyridine (70 mL) was then added. After 3 hours, the reaction was cooled to room temperature and the solution concentrated under reduced pressure. A mixture of ice/water was added to the residue and the mixture was stirred for 1 hour. The resulting off-white solid was filtered, washed with water and air dried to afford, after recrystallization from diethyl ether, 1-(3-phenanthryl)ethanone oxime (32 g).

### Step 2: 3-phenanthrylamine

To polyphosphoric acid (385 g) at 100° C. was added 1-(3-phenanthryl)ethanone oxime (32 g, 0.14 mol) from Step 1 above, over 30 minutes. The mixture was stirred at 100° C. for 2 hours, cooled to room temperature, followed by the addition of water and ice. The mixture was stirred for 30 minutes, filtered and the solids washed with water. The white solid obtained was then placed in a mixture of methanol (500 mL) and concentrated HCl (40 ml). The mixture was heated at reflux overnight, cooled to room temperature and concentrated under reduced pressure. A mixture of ethyl acetate and water was added to the residue and the resulting solution was made basic with 10 N KOH. The aqueous layer was extracted with ethyl acetate and the combined organic layers were washed successively with water and brine, then dried over Na₂SO₄ and filtered. The volatiles were removed under reduced pressure to afford 3-phenanthrylamine (25 g) as a beige solid.

### Step 3: 3-chlorophenanthrene

CuCl₂ (21 g) was dried under high vacuum at 115° C. for 90 minutes and then cooled to 65° C. Dry acetonitrile (250 mL) and t-butyl nitrite (26 g) were then added, followed by the addition of a solution of 3-phenanthrylamine (25 g) from Step 2 above, in acetonitrile (100 mL) over 30 minutes. The reaction was stirred for 45 minutes at 65° C., then cooled to room temperature. To the reaction mixture was then added HCl (1N, 1 L). The aqueous layer was extracted with methylene chloride and the combined organic layers were washed successively with water and brine, dried over Na₂SO₄ and filtered. The volatiles were removed under reduced pressure and the residue was purified by flash chromatography on silica (100% hexanes) to afford a white solid which was recrystallized from hexane to yield 3-chlorophenanthrene (14.4 g) as a white solid.

This phenanthrene could also be prepared by the following procedure:

### Step 3-a: 1-chloro-4-[2-phenylvinyl]benzene

To a solution of benzyltriphenylphosphonium chloride (58 g, 150 mmol) in DMF (500 mL) at 0° C., was added NaH (60% dispersion in oil, 7.2 g). The mixture was stirred at 0° C. for 1 hour, after which 4-chlorobenzaldehyde (21.1 g, 150 mmol) was added. The reaction mixture was warmed to room temperature and stirred for 1 hour. The reaction mixture was then poured into a mixture of water and Et₂O at 0° C. The aqueous layer was extracted with Et₂O. The combined organic layers were washed with brine, dried over Na₂SO₄ and filtered. The volatiles were removed under reduced pressure and the residue was purified by flash chromatography on silica (100% hexanes) to afford 1-chloro-4-[2-phenylvinyl]benzene (15.7 g, 49%) as a mixture of isomers.

### Step 3-b: 3-chlorophenanthrene

To a 2 L vessel equipped with a pyrex inner water-cooled jacket was added 1-chloro-4-[2-phenylvinyl]benzene (4 g) from Step 3-a above, in cyclohexane (2 L) and THF (7 mL). To this was added propylene oxide (20 equivalents) and iodine (1.5 equivalents). The stirred solution was first degassed by bubbling nitrogen through the mixture and was then exposed to UV light for 48 hours by inserting a 450 W medium pressure mercury lamp in the inner insert. The reaction was quenched with 10% Na₂S₂O₃ and the aqueous layer was extracted with diethyl ether. The combined organic layers were washed with brine, dried over Na₂SO₄ and the volatiles were removed under reduced pressure. This process was repeated 3 times on 4 g batches of 1-chloro-4-[2-phenylvinyl]benzene. The final residue obtained was recrystallized from hexanes to afford 3-chlorophenanthrene (11 g, 70%).

### Step 4: 3-chlorophenanthrene-9,10-dione

To a solution of 3-chlorophenanthrene (12.5 g, 58.7 mmol) from Step 3 above, in acetic acid (350 mL) was added CrO₃ (23.5 g, 0.23 mol). The reaction was stirred for 2 hours at 100° C., then cooled to room temperature and poured into water (2 L). The resulting suspension was stirred for 1 hour, filtered and the solids were washed with water. The solids were dried under high vacuum to afford 3-chlorophenanthrene-9,10-dione (12.5 g, 88%).

### Step 5: 6-chloro-2-(3,5-difluoropyridin-4-yl)-1H-phenanthro[9,10-d]imidazole

A mixture of 3-chlorophenanthrene-9,10-dione (150 mg, 0.62 mmol) from Step 4 above, ammonium acetate (478 mg, 6.2 mmol) and 3,5-difluoroisonicotinaldehyde (151 mg, 0.74 mmol) in acetic acid (5 mL) was heated at reflux for 4 hours. The mixture was then cooled to room temperature, poured into water and stirred for 10 minutes. The crude product was collected by filtration and then purified by flash chromatography on silica (5-20% acetone in methylene chloride) to afford 6-chloro-2-(3,5-difluoropyridin-4-yl)-1H-phenanthro[9,10-d]imidazole (115 mg, 50%). ¹H NMR δ (ppm) (400 MHz, DMSO-d₆): 14.0 (1H, bs), 9.0-8.87 (2H, m), 8.82 (2H, s), 8.6-8.5 (2H, m), 7.87-7.65 (3H, m).

### Example 221

### [6-chloro-2-(2,6-dibromophenyl)-1H-phenanthro[9,10-d]imidazol-9-yl](cyclopropyl)methanone

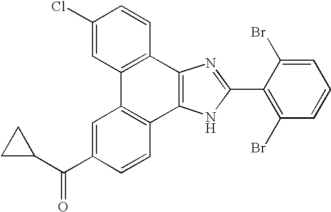

### Step 1: (6-chloro-3-phenanthryl)(cyclopropyl)methanol

To a −78° C. solution of 3-bromo-6-chlorophenanthrene (112 mg, 0.38 mmol) from Step 2 of Example 137, in THF (8 mL) was added methyllithium (1.6 M in Et₂O, 70 μL), followed by butyllithium (2.5 M in hexanes, 160 μL). The mixture was stirred at −78° C. for 15 minutes, after which cyclopropane carboxaldehyde (180 μL, 2.4 mmol) was added and the reaction mixture was stirred at −78° C. for 0.5 hours. The reaction was quenched with 25% NH₄OAc. The aqueous layer was extracted with ethyl acetate, the organic layer washed with brine, dried over Na₂SO₄, filtered and concentrated. The crude material was triturated in toluene to afford (6-chloro-3-phenanthryl)(cyclopropyl)methanol (60 mg, 55%) as a white solid.

### Step 2: (6-chloro-3-phenanthryl)(cyclopropyl)methanone

To a room temperature solution of (6-chloro-3-phenanthryl)(cyclopropyl)methanol (520 mg, 1.84 mmol) from Step 1 above, in CH₂Cl₂ (10 mL) and THF (2 mL) was added Dess Martin Periodinane reagent (780 mg, 1.84 mmol). The resulting yellow solution was stirred at room temperature for 2 hours. The reaction was quenched with 10% Na₂S₂O₃. The aqueous layer was extracted with ethyl acetate, the organic layer washed with brine, dried over Na₂SO₄, filtered and concentrated. The residue was purified by flash chromatography on silica (2-10% ethyl acetate in hexanes) to afford (6-chloro-3-phenanthryl)(cyclopropyl)methanone (467 mg, 90%) as a white solid.

### Step 3: 3-chloro-6-(cyclopropylcarbonyl)phenanthrene-9,10-dione

To a solution of (6-chloro-3-phenanthryl)(cyclopropyl)methanone (467 mg, 1.7 mmol) from Step 2 above, in acetic acid (10 mL) was added CrO₃ (670 mg, 6.71 mmol). The mixture was stirred at 70° C. for 20 minutes, then cooled to room temperature, poured into water and stirred for 45 minutes. The mixture was filtered and the solids obtained were washed with water and hexanes. The solids were dissolved in ethyl acetate, the organic layer washed with brine, dried over Na₂SO₄, filtered and concentrated to afford 3-chloro-6-(cyclopropylcarbonyl)phenanthrene-9,10-dione (493 mg, 95%) as an orange solid.

### Step 4: [6-chloro-2-(2,6-dibromophenyl)-1H-phenanthro[9,10-d]imidazol-9-yl](cyclopropyl)methanone

A solution of 3-chloro-6-(cyclopropylcarbonyl)phenanthrene-9,10-dione (493 mg, 1.58 mmol) from Step 3 above, ammonium acetate (2.45 g, 31.8 mmol) and 2,6-dibromobenzaldehyde (670 mg, 2.54 mmol) in acetic acid (40 mL) was heated at 70° C. for 20 minutes, after which it was cooled to room temperature, poured into water and stirred for 1 hour. The mixture was filtered and the solids washed with water and hexanes. A suspension of the solids in toluene was heated at reflux with a Dean Stark apparatus for 3 hours and then filtered. The solids obtained were dried under high vacuum to afford [6-chloro-2-(2,6-dibromophenyl)-1H-phenanthro[9,10-d]imidazol-9-yl](cyclopropyl)methanone (163 mg) as a yellow solid. ¹H NMR δ (ppm) (400 MHz, DMSO-d₆): 14.0 (1H, 2 singlets), 9.61 (1H, d), 9.29 (1H, d), 8.60 (1H, dd), 8.48-8.29 (2H, m), 7.92 (2H, d), 7.90-7.78 (1H, m), 7.50 (1H, t), 3.57-3.42 (1H, m), 1.21-1.09 (4H, m).

### Example 248

### 5-chloro-2-(2-chloro-6-fluorophenyl)-10-(methylsulfonyl)-1H-phenanthro[9,10-d]imidazole

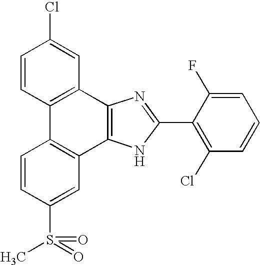

### Step 1: Methyl (2-bromo-5-chlorophenyl)acetate

To a solution of (2-bromo-5-chlorophenyl)acetic acid (4.99 g, 20 mmol) in methanol (100 mL) at 0° C. was slowly added thionyl chloride (2.2 mL, 30 mmol). The resulting solution was stirred at 0° C. for 10 minutes, then warmed to room temperature and stirred overnight. The reaction mixture was concentrated under reduced pressure. The crude material was dissolved in hexanes, filtered through Celite and the filtrate concentrated to yield methyl (2-bromo-5-chlorophenyl)acetate (5.13 g) as a colourless oil.

### Step 2: Methyl[4-chloro-4′-(methylthio)biphenyl-2-yl]acetate

A mixture of methyl (2-bromo-5-chlorophenyl)acetate (527 mg, 2 mmol from Step 1 above, [4-(methylthio)phenyl]boronic acid (320 mg, 2.2 mmol), cesium fluoride (608 mg, 4 mmol) and Pd(PPh₃)₄ (115 mg, 0.1 mmol) in DME (10 mL) was purged with nitrogen for 10 minutes, followed by heating at reflux for 4 hours. The reaction mixture was then cooled to room temperature, diluted with water and ethyl acetate. The organic layer was washed with water (3 times), dried over Na₂SO₄, filtered and concentrated. The material was purified by flash chromatography on silica (100% toluene) to provide methyl[4-chloro-4′-(methylthio)biphenyl-2-yl]acetate (554 mg).

### Step 3: [4-chloro-4′(methylthio)biphenyl-2-yl]acetic acid

To a solution of methyl[4-chloro-4′-(methylthio)biphenyl-2-yl]acetate (550 mg, 1.79 mmol) from Step 2 above, in THF (12 mL) and methanol (12 mL) was added sodium hydroxide (1N, 6 mL). The mixture was stirred at room temperature for 1.5 hours, then concentrated under reduced pressure to a small volume. The residue was diluted with water and acidified with HCl (1 N) to yield a precipitate which was filtered to afford [4-chloro-4′(methylthio)biphenyl-2-yl]acetic acid (475 mg) as a white solid.

### Step 4: 2-chloro-7-(methylthio)phenanthren-9-ol

To a solution of [4-chloro-4′(methylthio)biphenyl-2-yl]acetic acid (420 mg, 1.43 mmol) from Step 3 above, in 1,2-dichloroethane (7 mL) was added thionyl chloride (522 μL, 7.15 mmol). The reaction mixture was heated at 75° C. for 4 hours, after which it was concentrated at reduced pressure. The residue was co-evaporated with 1,2-dichloroethane (3 mL) to provide an orange syrup which was dissolved in 1,2-dichloroethane (12 mL). To this solution was added aluminium trichloride (287 mg, 2.15 mmol) at room temperature. The dark-red mixture was stirred at room temperature for 0.5 hours, then poured into ethyl acetate and water. The aqueous layer was extracted with ethyl acetate, the organic layer dried over Na₂SO₄, filtered and concentrated. The material was purified by flash chromatography on silica (100% toluene) to provide 2-chloro-7-(methylthio)phenanthren-9-ol (335 mg, 85%) as a light gray solid.

### Step 5: 2-chloro-7-(methylsulfonyl)phenanthren-9-ol

To a solution of 2-chloro-7-(methylthio)phenanthren-9-ol (148 mg, 0.54 mmol) from Step 4 above, in CH₂Cl₂ (25 mL) was added MCPBA (approx. 70%, 333 mg, 1.35 mmol). The resulting yellow solution was stirred at room temperature for 4 hours, after which CH₂Cl₂ (25 mL) was added, followed by Ca(OH)₂ (2 g). The mixture was stirred for 5 minutes, after which it was filtered through Celite. The filtrate was concentrated under reduced pressure, and the residue purified by flash chromatography on silica (40% ethyl acetate in toluene) to afford 2-chloro-7-(methylsulfonyl)phenanthren-9-ol (32 mg) as a yellow-orange solid.

### Step 6: 2-chloro-7-(methylsulfonyl)phenanthrene-9,10-dione

A mixture of 2-chloro-7-(methylsulfonyl)phenanthren-9-ol (32 mg, 0.1 mmol) Step 5 above and N,N′-Bis(salicylidene)ethylenediaminocobalt (II) hydrate (6 mg, 0.02 mmol) in DMF (3 mL) was stirred at room temperature overnight, with air bubbled through the reaction mixture. The mixture was then quenched with water, stirred for 5 minutes and the resulting solid precipitate filtered and washed with water to afford 2-chloro-7-(methylsulfonyl)phenanthrene-9,10-dione (23 mg) as a yellow solid.

Step 7: 5-chloro-2-(2-chloro-6-fluorophenyl)-10-(methylsulfonyl)-1H-phenanthro[9,10-d]imidazole

This imidazole was prepared as described in Step 5 of Example 182, substituting 2-chloro-7-(methylsulfonyl)phenanthrene-9,10-dione from Step 6 above for 3-chlorophenanthrene-9,10-dione, and substituting 2-fluoro-6-chlorobenzaldehyde for 3,5-difluoroisonicotinaldehyde, to afford 5-chloro-2-(2-chloro-6-fluorophenyl)-10-(methylsulfonyl)-1H-phenanthro[9,10-d]imidazole.

### Assays for Determining Biological Activity

### Inhibition of Prostaglandin E Synthase Activity

Compounds are tested as inhibitors of prostaglandin E synthase activity in microsomal prostaglandin e synthases, whole cell and in vivo assays. These assays measure prostaglandin E2 (PGE2) synthesis using either Enzymatic Immunoassay (EIA) or mass spectrometry. Cells used for microsomal preparation are CHO-K1 cells transiently transfected with plasmids encoding the human mPGES-1 cDNA. Cells used for cell-based experiments are human A549 (which express human mPGES-1). Guinea pigs are used to test the activity of selected compounds in vivo. In all these assays, 100% activity is defined as the PGE₂ production in vehicle-treated samples. IC₅₀ and ED₅₀ represent the concentration or dose of inhibitor required to inhibit PGE₂ synthesis by 50% as compared to the uninhibited control.

### Microsomal Prostaglandin E Synthase Assay

Prostaglandin E synthase microsomal fractions are prepared from CHO-K1 cells transiently transfected with plasmid encoding the human mPGES-1 cDNA. Microsomes are then prepared and the PGES assay begins with the incubation of 5 μg/ml microsomal PGES-1 with compound or DMSO (final 1%) for 20-30 minutes at room temperature. The enzyme reactions are performed in 200 mM KPi pH 7.0, 2 mM EDTA and 2.5 mM GSH-reduced form. The enzymatic reaction is then initiated by the addition of 1 μM final PGH2 substrate prepared in isopropanol (3.5% final in assay well) and incubated at room temperature for 30 seconds. The reaction is terminated by the addition of SnCl₂ in 1N HCl (1 mg/ml final). Measurement of PGE₂ production in the enzyme reaction aliquots is done by EIA using a standard commercially available kit (Cat #: 901-001 from Assay Designs).

Data from this assay for representative compounds is shown in the table below. The potency is expressed as IC₅₀ and the value indicated is an average of at least n=3.

| Ex. | h-CHO (nM) |
|---|---|
| 3 | 36 |
| 5 | 50 |
| 11 | 131 |
| 17 | 233 |
| 31 | 551 |
| 40 | 33 |
| 51 | 5.7 |
| 80 | 45 |
| 92 | 26 |
| 120 | 9.0 |
| 158 | 77 |
| 171 | 1.7 |
| 177 | 3.9 |
| 208 | 8.5 |
| 231 | 5.5 |
| 240 | 8.4 |

### Human A549 Whole Cell Prostaglandin E Synthase Assay

### Rationale

Whole cells provide an intact cellular environment for the study of cellular permeability and biochemical specificity of anti-inflammatory compounds such as prostaglandin E synthase inhibitors. To study the inhibitory activities of these compounds, human A549 cells are stimulated with 10 ng/ml recombinant human IL-1β for 24 hours. The production of PGE₂ and PGF_{2α} are measured by EIA at the end of the incubation as readouts for selectivity and effectiveness against mPGES-1-dependent PGE₂ production.

### Methods

Human A549 cells specifically express human microsomal prostaglandin E synthase-1 and induce its expression following treatment with IL-1β for 24 hours. 2.5×10⁴ cells seeded in 100 ul/well (96-well plate) and incubated overnight under standard conditions. 100 ul of cell culture media containing 10 ng/ml IL-1β is then added to the cells followed by the addition of either 2% FBS containing RPMI or 50% FBS containing RPMI. 2 μl of drugs or vehicle (DMSO) are then added and samples are mixed immediately. Cells are incubated for 24 hours and following the incubation 175 μl of medium is harvested and assayed for PGE₂ and PGF_{2α} contents by EIA.

### Human Whole Blood Prostaglandin E Synthase Assay

### Rationale

Whole blood provides a protein and cell-rich milieu for the study of biochemical efficacy of anti-inflammatory compounds such as prostaglandin E synthase inhibitors. To study the inhibitory activities of these compounds, human blood is stimulated with lipopolysaccharide (LPS) for 24 hours to induce mPGES-1 expression. The production of prostaglandin E2 (PGE₂) and thromboxane B2 (TxB₂) are measured by EIA at the end of the incubation as readouts for selectivity and effectiveness against mPGES-1-dependent PGE₂ production.

### Methods

Human whole blood assays for mPGES-1 activity reported (Brideau, et al., Inflamm. Res., vol. 45, p. 68, 1996) are performed as described below.

Freshly isolated venous blood from human volunteers is collected in heparinized tubes. These subjects have no apparent inflammatory conditions and have not taken any NSAIDs for at least 7 days prior to blood collection. 250 μl of blood is pre-incubated with 1 ul vehicle (DMSO) or 1 ul of test compound. Bacterial LPS at 100 g/ml (E. Coli serotype 0111:B4 diluted in 0.1% w/v bovine serum albumin in phosphate buffered saline) is then added and samples are incubated for 24 hours at 37° C. Unstimulated control blood at time zero (no LPS) is used as blank. At the end of the 24 hr incubation, the blood is centrifuged at 3000 rpm for 10 min at 4° C. The plasma is assayed for PGE₂ and TxB₂ using an EIA kit as indicated above.

### In Vivo Determination of Anti-Inflammatory Activity

### Rationale

The whole animal provides an integrated physiological system to confirm the anti-inflammatory activity of test compounds characterized in vitro. To determine the activity of prostaglandin E synthase inhibitors in vivo, animals are dosed with compounds either prior or after the inflammatory stimulus, LPS. LPS is injected into the hind paw of guinea pigs and hyperalgesia measurements are recorded 4.5 and/or 6 hrs after the injection.

### Formulation of Test Compounds for Oral Dosage

Test compound was ground and made amorphous using a ball milling system. The compound was placed in an agate jar containing agate balls and spun at high speed for 10 minutes in an apparatus such as the Planetary Micro Mill Pulverisette 7 system. The jar was then opened and 0.5% methocel solution was added to the ground solid. This mixture was spun again at high speed for 10 minutes. The resulting suspension was transferred to a scintillation vial, diluted with the appropriate amount of 0.5% methocel solution, sonicated for 2 minutes and stirred until the suspension was homogeneous. Alternatively, the test compound can be formulated using amorphous material obtained by any suitable chemical or mechanical technique. This amorphous solid is then mixed and stirred for a certain period of time, such as 12 hours, with a suitable vehicle, such as 0.5% methocel with 0.02 to 0.2% of sodium dodecylsulfate, prior to dosage.

### Methods

Male Hartley guinea pigs, weighing 200-250 grams were used. LPS (30 mg/kg) is injected sub-plantarly into the left hind paw of the guinea pig to produce hyperalgesia in the injected paw. Rectal temperature and paw withdrawal latency, a measure of hypersensitivity to pain (hyperalgesia), are taken prior to LPS injection and used as the baseline. Paw withdrawal latency is determined using the thermal hyperalgesia instrument (Ugo Basile Corp.). During this determination, animals are placed in an 8″×8″ plexiglass holding box atop of a glass base. A mild (223 mW/cm²) infrared light is directed toward the underside of the hind paw. The time it takes for the animal to remove its paw (indication that it feels the pain caused by the heat) is recorded. The infrared light immediately shuts off when the animal withdraws its paw from the area. The light will also shut off automatically when the time reaches 20 seconds.

### Predose Paradigm:

Test compounds are orally dosed at 5 ml/kg using an 18-gauge feeding needle. LPS (serotype 0111:B4, 10 μg) or 0.9% saline is injected into the plantar region of the left hind paw at a volume of 100 μl using a 26 gauge needle 1 hour following compound administration. Rectal temperature and thermal paw withdrawal latency are taken 4.5 hours after LPS administration. The animals are euthanized following the measurements using CO₂ and lumbar spinal cord, hind paw and blood samples collected.

### Reversal Paradigm:

Thermal paw withdrawal of each animal is determined before and 3 hours following sub-plantar injection of LPS. Animals which have received LPS and do not show a decrease in withdrawal latency at the 3 hour time point will be removed from study and euthanized. Test compounds are dosed p.o. at 5 ml/kg immediately following the thermal paw withdrawal measurement. Thermal withdrawal latency is taken 1.5 and 3 hours following compound administration (4.5 and 6 hours post-LPS administration). After the final reading, the animals are euthanized using CO2 and lumbar spinal cord and blood samples collected for prostaglandin determination by mass spectrometry and drug level, respectively.

The invention also encompasses a compound represented by Formula I

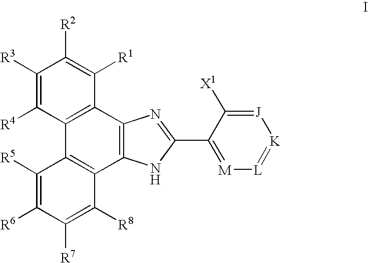

or a prodrug thereof, or a pharmaceutically acceptable salt of said compound or prodrug, wherein:
J is selected from the group consisting of —C(X²)— and —N—,
K is selected from the group consisting of C(X³)— and —N—,
L is selected from the group consisting of —C(X⁴)— and —N—, and
M is selected from the group consisting of —C(X⁵)— and —N—,
with the proviso that at least one of J, K, L or M is other than —N—, and with the proviso that when J is —C(X²)—, K is —C(X³)—, L is —C(X⁴)—, M is —C(X⁵)— and X⁵ is H, then at least one of R³ and R⁶ is other than H;
X¹ is selected from the group consisting of: (1) F; (2) Cl; (3) Br; (4) I; (5) —N₃; (6) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy group; (7) C₁₋₄alkoxy; (8) NR⁹R¹⁰—C(O)—C₁₋₄alkyl-O—; (9) C₁₋₄alkyl-S(O)ₖ—; (10) —NO₂; (11) C₃₋₆cycloalkyl, (12) C₃₋₆cycloalkoxy; (13) phenyl, (14) carboxy; and (15) C₁₋₄alkyl-O—C(O)—;
X², X³ and X⁴ are independently selected from the group consisting of: (1) H; (2) F; (3) Cl; (4) Br; (5) I; (6) —OH; (7) —N₃; (8) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (9) C₁₋₄alkoxy; (10) NR⁹R¹⁰—, NR⁹R¹⁰—C(O)—C₁₋₄alkyl-O— or NR⁹R¹⁰—C(O)—; (11) C₁₋₄alkyl-S(O)ₖ—; (12) —NO₂; (13) C₃₋₆cycloalkyl, (14) C₃₋₆cycloalkoxy; (15) phenyl, (16) carboxy, (17) C₁₋₄alkyl-O—C(O)— and (18) —CN;
X⁵ is selected from the group consisting of: (1) H; (2) F; (3) Cl; (4) Br; (5) I; (6) —OH; (7) —N₃; (8) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (9) C₁₋₄alkoxy; (10) NR⁹R¹⁰—, NR⁹R¹⁰—C(O)—C₁₋₄alkyl-O— or NR⁹R¹⁰—C(O)—; (11) C₁₋₄alkyl-S(O)ₖ—; (12) —NO₂; (13) C₃₋₆cycloalkyl, (14) C₃₋₆cycloalkoxy; (15) phenyl, (16) carboxy, and (17) C₁₋₄alkyl-O—C(O)—;
R¹, R², R¹³, R⁴, R⁵, R⁶, R⁷ and R⁸ are independently selected from the group consisting of: (1) H; (2) F; (3) Cl; (4) Br; (5) I; (6) —CN; (7) C₁₋₁₀alkyl or C₂₋₁₀alkenyl, wherein one or more of the hydrogen atoms attached to said C₁₋₁₀alkyl or C₂₋₁₀alkenyl may be replaced with a fluoro atom, or two hydrogen on adjacent carbon atoms may be joined together and replaced with —CH₂— to form a cyclopropyl group, or two hydrogen atoms on the same carbon atom may be replaced and joined together to form a spiro C₃₋₆cycloalkyl group, and wherein said C₁₋₁₀ alkyl or C₂₋₁₀alkenyl may be optionally substituted with one to three substituents independently selected from the group consisting of: —OH, acetyl, methoxy, ethenyl, R¹¹—O—C(O)—, R³⁵—N(R³⁶)—, R³⁷—N(R³⁸)—C(O)—, cyclopropyl, pyrrolyl, imidiazolyl, pyridyl and phenyl, said pyrrolyl, imidiazolyl, pyridyl and phenyl optionally substituted with C₁₋₁₄alkyl or mono-hydroxy substituted C₁₋₄alkyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C; (18) (CH₃)C═N—OH or —(CH₃)C═N—OCH₃; (19) R³⁴—O—C(O)—; (20) R³⁹—C(O)—O—; and (21) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with a substituent independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₄alkyl optionally substituted with 1 to 3 groups independently selected from halo and hydroxy;
each Z is independently selected from the group consisting of: (1) H; (2) C₁₋₆alkyl, wherein one or more of the hydrogen atoms attached to said C₁₋₆alkyl may be replaced with a fluoro atom, and wherein C₁₋₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or —(CH₃)C═N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and (2) C₁₋₄alkyl;
each R¹¹, R¹², R¹³, R¹⁴, R¹⁶, R²³, R²⁵, R³⁰, R³¹, R³⁴ and R³⁹ is independently selected from the group consisting of: (1) H; (2) C₁₋₄alkyl, (3) C₃₋₆cycloalkyl; (4) C₃₋₆cycloalkyl-C₁₋₄alkyl- (5) phenyl, (6) benzyl; and (7) pyridyl; said C₁₋₄alkyl, C₃₋₆cycloalkyl, C₃₋₆cycloalkyl-C₁₋₄alkyl-, phenyl, benzyl and pyridyl may each be optionally substituted with 1 to 3 substituents independently selected from the group consisting of: OH, F, Cl, Br and I, and wherein said C₁₋₄alkyl may be further substituted with oxo or methoxy or both;
each R¹⁷, R¹⁸, R¹⁹, R²⁰, R²¹, R²², R²⁶, R²⁷, R²⁸, R²⁹, R³⁵, R³⁶, R³⁷ and R³⁸ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹, R³⁵ and R³⁶, and R³⁷ and R³⁸ may be joined together with the nitrogen atom to which they are attached to form a monocyclic ring of 5 or 6 carbon atoms, optionally containing one or two atoms independently selected from —O—, —S(O)ₖ— and —N(R³²)—; and each k is independently 0, 1 or 2.

The invention also encompasses a sub-class of compounds of Formula A wherein: R³ and R⁶ are independently selected from the group consisting of: hydrogen, fluoro, chloro, bromo, iodo, cyano, methyl, methoxy, ethyl, vinyl, cyclopropyl, propyl, butyl, —CO₂i-Pr, —CO₂CH₃, —SO₂CF₃, 3-pyridyl, acetyl,

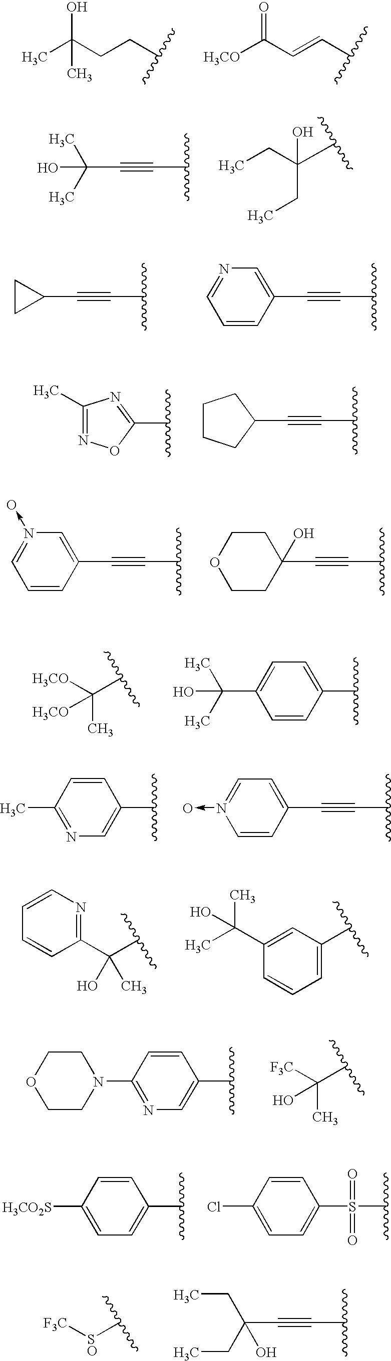

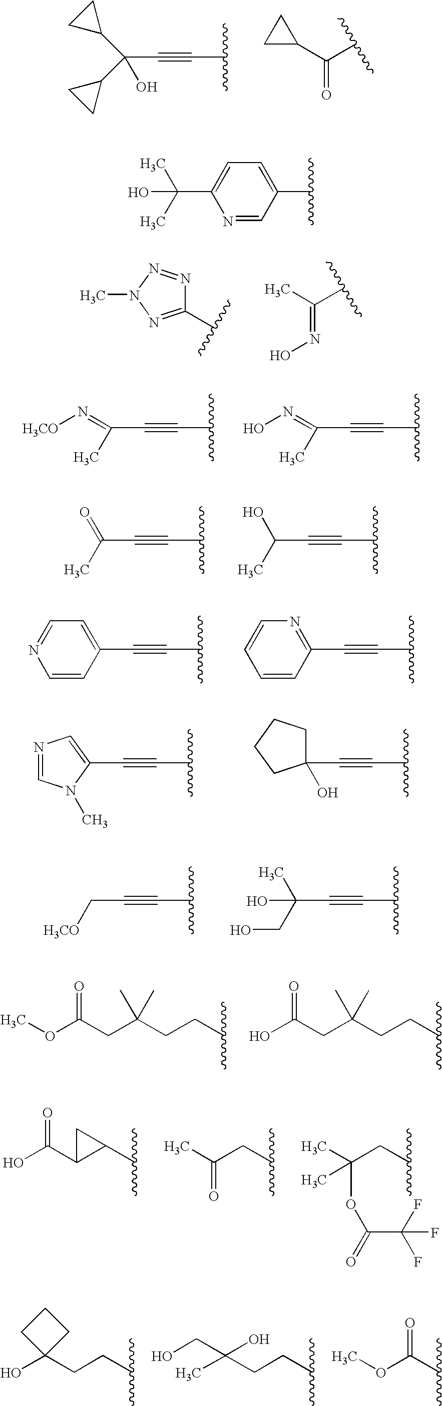

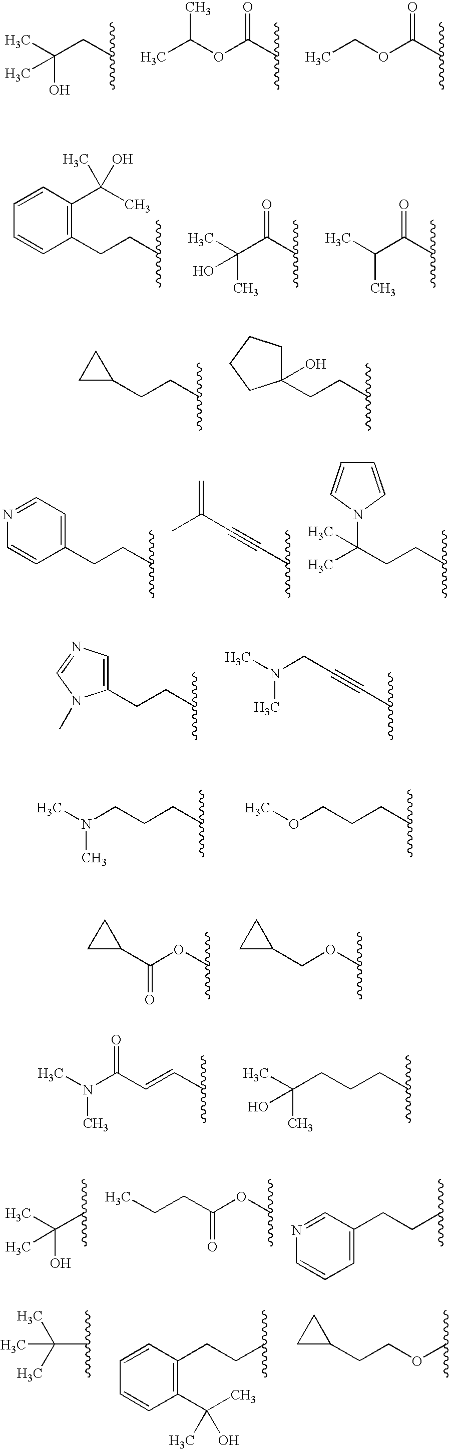

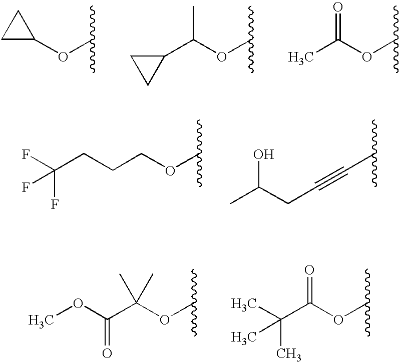

with the proviso that at least one of R³ or R⁶ is other than hydrogen.

What is claimed is:

1. A compound represented by Formula I 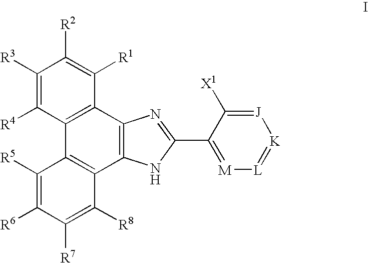
or a prodrug thereof, or a pharmaceutically acceptable salt of said compound or prodrug, wherein:
J is selected from the group consisting of —C(X²)— and —N—,
K is selected from the group consisting of —C(X³)— and —N—,
L is selected from the group consisting of —C(X⁴)— and —N—, and
M is selected from the group consisting of —C(XS)— and —N—,
with the proviso that at least one of J, K, L or M is other than —N—, and with the proviso that when J is —C(X²)—, K is —C(X³)—, L is —C(X⁴)—, M is —C(X⁵)— and X⁵ is H, then at least one of R³ and R⁶ is other than H;
X¹ is selected from the group consisting of: (1) F; (2) Cl; (3) Br; (4) I; (5) —N₃; (6) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy group; (7) C₁₋₄alkoxy; (8) NR⁹R¹⁰—C(O)—C₁₋₄alkyl-O—; (9) C₁₋₄alkyl-S(O)ₖ—; (10) —NO₂; (11) C₃₋₆cycloalkyl, (12) C₃₋₆cycloalkoxy; (13) phenyl, (14) carboxy; and (15) C₁₋₄alkyl-O—C(O)—;
X², X³ and X⁴ are independently selected from the group consisting of: (1) H; (2) F; (3) Cl; (4) Br; (5) I; (6) OH; (7) —N₃; (8) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (9) C₁₋₄alkoxy; (10) NR⁹R¹⁰—, NR⁹R¹⁰—C(O)—C₁₋₄alkyl-O— or NR⁹R¹⁰—C(O)—;
(11) C₁₋₄allyl-S(O)ₖ—; (12) —NO₂; (13) C₃₋₆cycloalkyl, (14) C₃₋₆cycloalkoxy; (15) phenyl, (16) carboxy, (17) C₁₋₄alkyl-O—C(O)— and (18) —CN;
X⁵ is selected from the group consisting of: (1) H; (2) F; (3) Cl; (4) Br; (5) I; (6) —OH; (7) —N₃; (8) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (9) C₁₋₄alkoxy; (10) NR⁹R¹⁰—, NR⁹R¹⁰—C(O)—C₁₋₄alkyl-O— or NR⁹R¹⁰—C(O)—; (11) C₁₋₄alkyl-S(O)ₖ—; (12) —NO₂; (13) C₃₋₆cycloalkyl, (14) C₃₋₆cycloalkoxy; (15) phenyl, (16) carboxy, and (17) C₁₋₄alkyl-O—C(O)—;
R¹, R², R³, R⁴, R⁵, R⁶, R⁷ and R⁸ are independently selected from the group consisting of: (1) H; (2) F; (3) Cl; (4) Br; (5) I; (6) —CN; (7) C₁₋₁₀alkyl or C₂₋₁₀alkenyl, wherein one or more of the hydrogen atoms attached to said C₁₋₁₀alkyl or C₂₋₁₀alkenyl may be replaced with a fluoro atom, or two hydrogen on adjacent carbon atoms may be joined together and replaced with —CH₂— to form a cyclopropyl group, or two hydrogen atoms on the same carbon atom may be replaced and joined together to form a spiro C₃₋₆cycloalkyl group, and wherein said C₁₋₁₀alkyl or C₂₋₁₀alkenyl may be optionally substituted with one to three substituents independently selected from the group consisting of: —OH, acetyl, methoxy, ethenyl, R¹¹—O—C(O)—, R³⁵—N(R³⁶)—, R³⁷—N(R³⁸)—C(O)—, cyclopropyl, pyrrolyl, imidiazolyl, pyridyl and phenyl, said pyrrolyl, imidiazolyl, pyridyl and phenyl optionally substituted with C₁₋₄alkyl or mono-hydroxy substituted C₁₋₄alkyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C; (18) —(CH₃)C═N—OH or —(CH₃)C═N—OCH₃; (19) R³⁴—O—C(O)—; (20) R³⁹—C(O)—O—; and (21) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with a substituent independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₄alkyl optionally substituted with 1 to 3 groups independently selected from halo and hydroxy;
each Z is independently selected from the group consisting of: (1) H; (2) C₁₋₆alkyl, wherein one or more of the hydrogen atoms attached to said C₁₋₆alkyl may be replaced with a fluoro atom, and wherein C₁₋₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or (CH₃)C═N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and (2) C₁₋₄alkyl;
each R¹¹, R¹², R¹³, R¹⁴, R¹⁶, R²³, R²⁵, R³⁰, R³¹, R³⁴ and R³⁹ is independently selected from the group consisting of: (1) H; (2) C₁₋₄alkyl, (3) C₃₋₆cycloalkyl; (4) C₃₋₆cycloalkyl-C₁₋₄alkyl- (5) phenyl, (6) benzyl; and (7) pyridyl; said C₁₋₄alkyl, C₃₋₆cycloalkyl, C₃₋₆cycloalkyl-C₁₋₄alkyl-, phenyl, benzyl and pyridyl may each be optionally substituted with 1 to 3 substituents independently selected from the group consisting of: OH, F, Cl, Br and I, and wherein said C₁₋₄alkyl may be further substituted with oxo or methoxy or both;
each R¹⁷, R¹⁸, R¹⁹, R²⁰, R²¹, R²², R²⁶, R²⁷, R²⁸, R²⁹, R³⁵, R³⁶, R³⁷ and R³⁸ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹, R³⁵ and R³⁶, and R³⁷ and R³⁸ may be joined together with the nitrogen atom to which they are attached to form a monocyclic ring of 5 or 6 carbon atoms, optionally containing one or two atoms independently selected from —O—, —S(O)ₖ— and —N(R³²)—; and
each k is independently 0, 1 or 2.

2. A compound according to claim 1 represented by Formula I 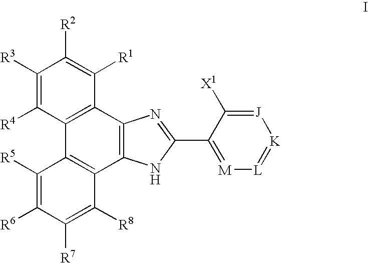
or a prodrug thereof, or a pharmaceutically acceptable salt of said compound or prodrug, wherein:
J is selected from the group consisting of —C(X²)— and —N—,
K is selected from the group consisting of —C(X³)— and —N—,
L is selected from the group consisting of —C(X⁴)— and —N—, and
M is selected from the group consisting of —C(X⁵)— and —N—,
with the proviso that at least one of J, K, L or M is other than —N—, and with the proviso that when J is —C(X²)—, K is C(X³)—, L is —C(X⁴)—, M is —C(X⁵)— and X⁵ is H, then at least one of R³ and R⁶ is other than H;
X¹ is selected from the group consisting of: (1) F; (2) Cl; (3) Br; (4) I; (5) —N₃; (6) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy group; (7) C₁₋₄alkoxy; (8) NR⁹R¹⁰—C(O)—C₁₋₄alkyl-O—; (9) C₁₋₄alkyl-S(O)ₖ—; (10) —NO₂; (11) C₃₋₆cycloalkyl, (12) C₃₋₆cycloalkoxy; (13) phenyl, (14) carboxy; and (15) C₁₋₄alkyl-O—C(O)—;
X², X³, X⁴ and X⁵ are independently selected from the group consisting of: (1) H; (2) F; (3) Cl; (4) Br; (5) I; (6) —OH; (7) —N₃; (8) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (9) C₁₋₄alkoxy; (10) NR⁹R¹⁰—, NR⁹R¹⁰—C(O)—C₁₋₄alkyl-O— or NR⁹R¹⁰—C(O)—; (11) C₁₋₄alkyl-S(O)ₖ—; (12) —NO₂; (13) C₃₋₆cycloalkyl, (14) C₃₋₆cycloalkoxy; (15) phenyl, (16) carboxy; and (17) C₁₋₄alkyl-O—C(O)—;
R¹, R², R³, R⁴, R⁵, R⁶, R⁷ and R⁸ are independently selected from the group consisting of: (1) H; (2) F; (3) Cl; (4) Br; (5) I; (6) —CN; (7) C₁₋₆alkyl or C₂₋₆alkenyl, wherein one or more of the hydrogen atoms attached to said C₁₋₆alkyl or C₂₋₆alkenyl may be replaced with a fluoro atom, and wherein said C₁₋₆alkyl or C₂₋₆alkenyl may be optionally substituted with one to three substituents independently selected from the group consisting of: —OH, methoxy, R¹¹—O—C(O)—, cyclopropyl, pyridyl and phenyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C; (18) —(CH₃)C═N—OH or (CH₃)C═N—OCH₃; and (19) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with 1 to 3 substituents independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₄alkyl and phenyl optionally substituted with 1 to 3 groups independently selected from halo and hydroxy; or R⁵ and R⁶ or R⁷ and R⁸ may be joined together with the carbon atoms to which they are attached to form phenyl;
each Z is independently selected from the group consisting of: (1) H; (2) C₁₋₆alkyl, wherein one or more of the hydrogen atoms attached to said C₁₋₆alkyl may be replaced with a fluoro atom, and wherein C₁₋₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or —(CH₃)C═N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and (2) C₁₋₄alkyl;
each R¹¹, R¹², R¹³, R¹⁴, R¹⁶, R²³, R²⁵, R³⁰ and R³¹ is independently selected from the group consisting of: (1) H; (2) C₁₋₄alkyl, (3) C₃₋₆cycloalkyl; (4) phenyl, (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br, I and methyl;
each R¹⁷, R¹⁸, R¹⁹, R²⁰, R²¹, R²², R²⁶, R²⁷, R²⁸ and R²⁹ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹ may be joined together with the nitrogen atom to which they are attached to form a monocyclic ring of 5 or 6 carbon atoms, optionally containing one or two atoms independently selected from —O—, —S(O)ₖ— and —N(R³²)—; and
each k is independently 0, 1 or 2.

3. A compound according to claim 1 according to Formula A 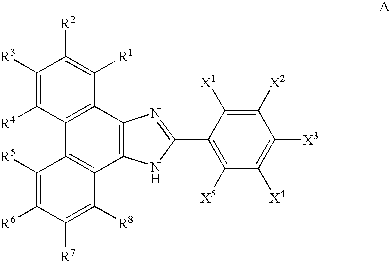
or a prodrug thereof, or a pharmaceutically acceptable salt of said compound or prodrug.

4. The compound according to claim 3 wherein:
X¹ is selected from the group consisting of: (1) F; (2) Cl; (3) Br; and (4) I; and
X², X³, X⁴ and X⁵ are independently selected from the group consisting of: (1) H; (2) F; (3) Cl; (4) Br; and (5) I.

5. The compound according to claim 3, wherein X², X³ and X⁴ are H, and X⁵ is other than H.

6. The compound according to claim 5, wherein X¹ and X⁵ are the same and selected from the group consisting of: (1) F; (2) Cl; (3) Br; and (4) I.

7. The compound according to claim 3, wherein at least one of R¹ or R⁸ is other than H.

8. The compound according to claim 3 wherein at least one of R² or R⁷ is other than H.

9. The compound according to claim 3 wherein at least one of R⁴ or R⁵ is other than H.

10. The compound according to claim 3 wherein:
at least one of R³ or R⁶ is other than H; and
R¹, R², R⁴, R⁵, R⁷ and R⁸ are H.

11. The compound according to claim 10, wherein R³ and R⁶ are both other than H.

12. The compound according to claim 11, wherein:
one of R³ or R⁶ is independently selected from the group consisting of: F, Cl, Br, and I; and
the other of R³ or R⁶ is Z-C≡C.

13. The compound according to claim 10, wherein: R³ and R⁶ are independently selected from the group consisting of: hydrogen, fluoro, chloro, bromo, iodo, cyano, methyl, ethyl, vinyl, cyclopropyl, —CO₂i-Pr, —CO₂CH₃, —SO₂CF₃, 3-pyridyl, acetyl, 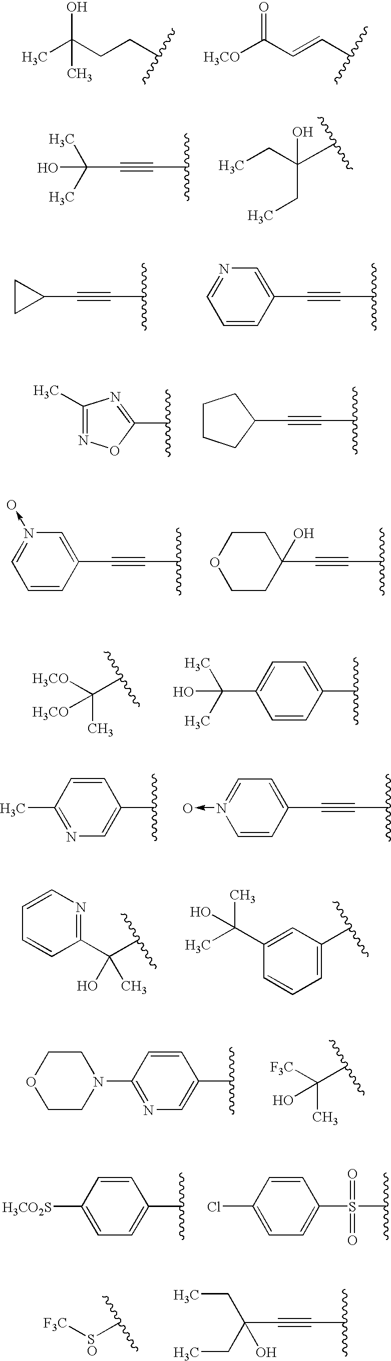 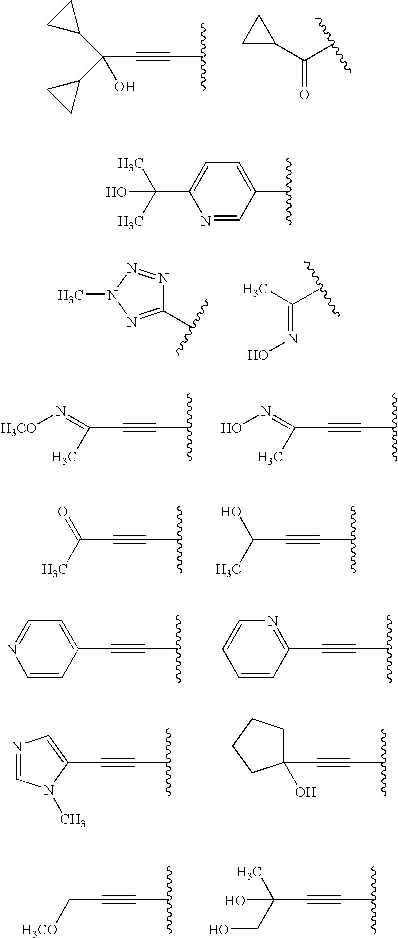
with the proviso that at least one of R³ or R⁶ is other than H.

14. The compound according to claim 2 according to Formula B: 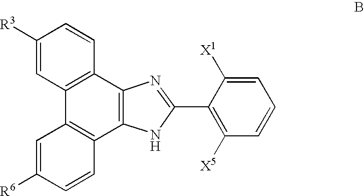
or a prodrug thereof, or a pharmaceutically acceptable salt of said compound or prodrug, wherein:
X¹ and X⁵ are independently selected from the group consisting of: (1) F; (2) Cl; (3) Br; and (4) I.

15. The compound according to claim 14 wherein:
one of R³ or R⁶ is independently selected from the group consisting of: F, Cl, Br, and I; and
the other of R³ or R⁶ is Z-C≡C.

16. A prodrug of a compound according to claim 1 of Formula C 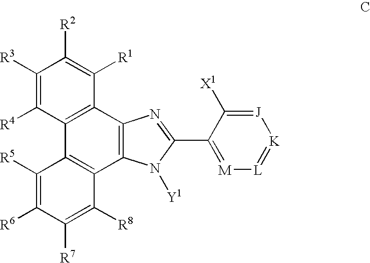
or a pharmaceutically acceptable salt thereof, wherein:
Y¹ is selected from the group consisting of: (1) C₁₋₆alkyl; (2) PO₄—C₁₋₄alkyl-; (3) C₁₋₄alkyl-C(O)—O—CH₂—, wherein the C₁₋₄alkyl portion is optionally substituted with R³³—O—C(O)—; and (4) C₁₋₄alkyl-O—C(O)—; and
R³³ is selected from the group consisting of: (1) H; (2) C₁₋₄alkyl, (3) C₃₋₆cycloalkyl; (4) phenyl; (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br and I.

17. A compound according to claim 1 selected from one of the following tables:
TABLE 1

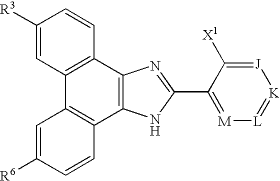

| R³/R⁶ | R⁶/R³ | X¹ | J | K | L | M |
|---|---|---|---|---|---|---|
| H | H | Cl | CH | CH | CH | CF |
| H | H | Cl | CH | N | CH | CCl |
| H | H | CH₃ | CH | CCH₃ | CH | CCH₃ |
| Br | Br | Cl | CH | CH | CH | CF |
| H | H | Br | CH | CH | CH | N |
| Br | CO₂H | Cl | CH | CH | CH | CF |
| H | H | Br | CH | N | CH | CH |
| Br | H | Cl | CH | CH | CH | CF |
| CO₂CH₃ | CO₂CH₃ | Cl | CH | CH | CH | CF |
| Br | Br | CO₂CH₃ | CH | CH | CH | CH |
| H | H | Cl | N | CH | CH | CH |
| Br | Br | CO₂Na | CH | CH | CH | CH |
| Br | Br | CH₃ | CH | CH | CH | CH |
| Br | Br | 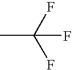 | CH | CF | CH | CH |
| Br | Br | CL | N | CH | CH | CH |
| Br | Br | NO₂ | CH | CH | CH | CH |
| Cl | Br | F | CH | CH | CH | C(CONH₂) |
| 3-pyridyl | 3-pyridyl | Cl | CH | CH | CH | CF |
| Br | 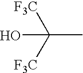 | Cl | CH | CH | CH | CF |
| Cl | Br | F | CH | CH | CH | C(COCH₃) |
| Br | Br | H | CH | C(SCF₃) | CH | CH |
| Br | Br | CF₃ | CH | CH | CH | CH |
| Br | Br | I | CH | CH | CH | CH |
| Br | Br | CH₃ | CH | CCH₃ | CH | CH |
| Br | Br | Br | CH | CH | CH | CH |
| Br | Br | CF₃ | CH | CH | CH | CF |
| CN | CN | CF₃ | CH | CF | CH | CH |
| CN | Br | Cl | CH | CF | CH | CH |
| Br | Br | CH₃ | CH | C(N(CH₃)₂) | CH | CH |
| H | 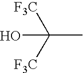 | Cl | CH | CH | CH | CF |
| Br | H | Cl | CH | CF | CH | CH |
| CN | Br | 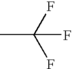 | CH | CF | CH | CH |
| CH₃ | Br | Cl | CH | CH | CH | CF |
| OCH₃ | Br | Cl | CH | CH | CH | CF |
| Br | Br | Cl | CH | CF | CH | CH |
| H | CO₂H | Cl | CH | CH | CH | CF |
| 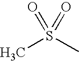 | Br | Cl | CH | CH | CH | CF |
| H | 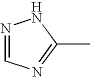 | Cl | CH | CH | CH | CF |
| H | 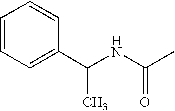 | Cl | CH | CH | CH | CF |
| Br | 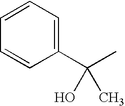 | Cl | CH | CH | CH | CF |
| 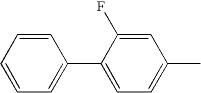 | H | Cl | CH | CH | CH | CF |
| Br | 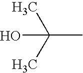 | Cl | CH | CH | CH | CF |
| Br | 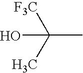 | Cl | CH | CH | CH | CF |
| H | H | Cl | CH | CH | CH | CCl |
| H | H | F | CH | CH | CH | CF |
| H | H | F | CF | CH | CH | CF |
| 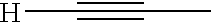 | H | F | CH | CH | CH | CCl |
| i-Pr | Br | Cl | CH | CH | CH | CF |
| 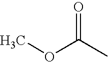 | 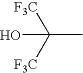 | Cl | CH | CH | CH | CF |
| 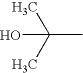 | 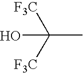 | Cl | CH | CH | CH | CF |
| CH₃ | H | Cl | CH | CH | CH | CF |
| CH₃ | 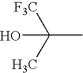 | Cl | CH | CH | CH | CF |
| CH₃ | CH₃ | Cl | CH | CH | CH | CF |
| 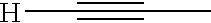 | 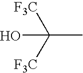 | F | CH | CH | CH | CCl |
| 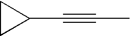 | 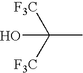 | F | CH | CH | CH | CCl |
| 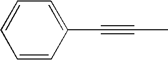 | 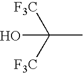 | F | CH | CH | CH | CC1 |
| CHO | 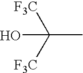 | Cl | CH | CH | CH | CF |
| 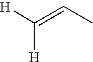 | 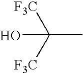 | F | CH | CH | CH | CCl |
| 3-pyridyl | 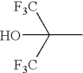 | Cl | CH | CH | CH | CF |
| Br | 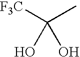 | Cl | CH | CH | CH | CF |
| 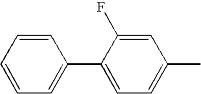 | 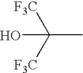 | Cl | CH | CH | CH | CF |
| 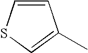 | 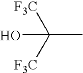 | Cl | CH | CH | CH | CF |
| Br | H | CF₃ | CH | CH | CH | CF |
| Br | 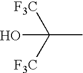 | CF₃ | CH | CH | CH | CF |
| OCH₃ | 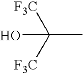 | Cl | CH | CH | CH | CF |
| 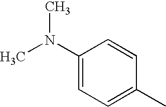 | 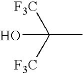 | Cl | CH | CH | CH | CF |
| I | 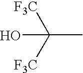 | F | CH | CH | CH | CCl |
| 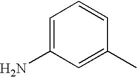 | 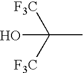 | Cl | CH | CH | CH | CF |
| 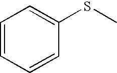 | 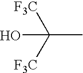 | F | CH | CH | CH | CCl |
| 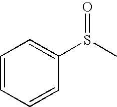 | 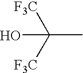 | F | CH | CH | CH | CCl |
| 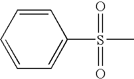 | 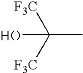 | F | CH | CH | CH | CCl |
| 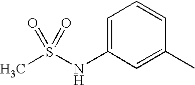 | 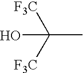 | Cl | CH | CH | CH | CF |
| 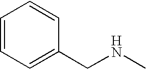 | 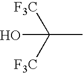 | Cl | CH | CH | CH | CF |
| Br | 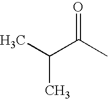 | Cl | CH | CH | CH | CF |
| Br | 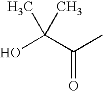 | Cl | CH | CH | CH | CF |
| Br | 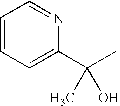 | Cl | CH | CH | CH | CF |
| 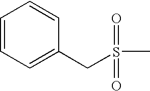 | 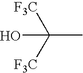 | F | CH | CH | CH | CCl |
| 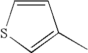 | 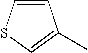 | F | CH | CH | CH | CCl |
| F | H | Cl | CH | CH | CH | CF |
| 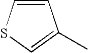 | Br | F | CH | CH | CH | CCl |
| 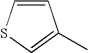 | 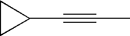 | F | CH | CH | CH | CCl |
| 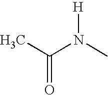 | H | Cl | CH | CH | CH | CF |
| 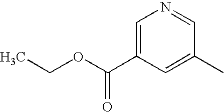 | 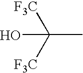 | Cl | CH | CH | CH | CF |
| 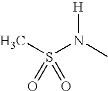 | H | Cl | CH | CH | CH | CF |
| —SO₂NH₂ | H | F | CH | CH | CH | CCl |
| 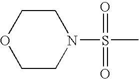 | H | F | CH | CH | CH | CCl |
| 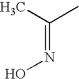 | Br | Cl | CH | CH | CH | CF |
| 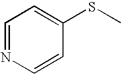 | 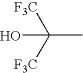 | F | CH | CH | CH | CCl |
| 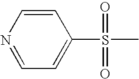 | 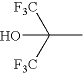 | F | CH | CH | CH | CCl |
| 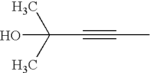 | 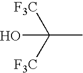 | F | CH | CH | CH | CCl |
| 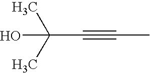 | Br | F | CH | CH | CH | CCl |
| 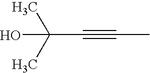 | 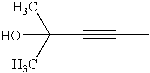 | F | CH | CH | CH | CCl |
| Cl | H | Cl | CH | CH | CH | CF |
| Br | 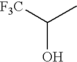 | F | CH | CH | CH | CCl |
| 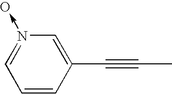 | 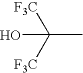 | F | CH | CH | CH | CCl |
| 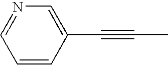 | 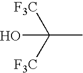 | F | CH | CH | CH | CCl |
| H | H | H | CH | CH | CH | N |
| 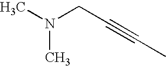 | 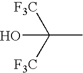 | F | CH | CH | CH | CCl |
| 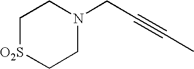 | 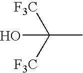 | F | CH | CH | CH | CCl |
| H | H | OCH₃ | CH | CH | CH | C(OCH₃) |
| Br | Br | Br | CH | CH | CH | CF |
| Cl | Br | Cl | CH | CH | CH | CF |
| Cl | 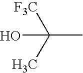 | Cl | CH | CH | CH | CF |
| CN | H | F | CH | CH | CH | CCl |
| CN | 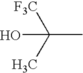 | Cl | CH | CH | CH | CF |
| Br | Br | I | CH | CH | CH | CF |
| Cl | 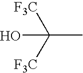 | Cl | CH | CH | CH | CF |
| H | H | 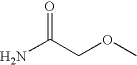 | CH | CH | CH | CH |
| Br | Br | 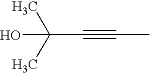 | CH | CH | CH | CF |
| Cl | 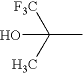 | F | CH | CH | CH | CH |
| OCH₃ | H | Cl | CH | CH | CH | CF |
| H | H | Br | CH | CH | CH | CBr |
| H | H | F | CH | CH | CH | CBr |
| Cl | H | Cl | CH | CH | CH | COH |
| Br | 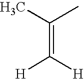 | Cl | CH | CH | CH | CF |
| Cl | H | 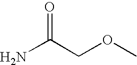 | CH | CH | CH | CCl |
| Cl | Br | Br | CH | CH | CH | CF |
| 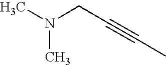 | Br | Cl | CH | CH | CH | CF |
| 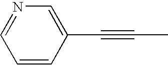 | Br | Cl | CH | CH | CH | CF |
| Br | 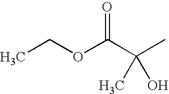 | Cl | CH | CH | CH | CF |
| 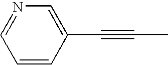 | 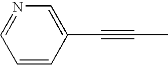 | Cl | CH | CH | CH | CF |
| Cl | Cl | Cl | CH | CH | CH | CF |
| H | H | Br | CH | N | CH | CBr |
| H | H | F | CH | N | CH | CH |
| Br | 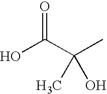 | Cl | CH | CH | CH | CF |
| 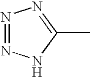 | Cl | Cl | CH | CH | CH | CF |
| 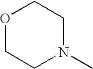 | H | Cl | CH | CH | CH | CF |
| Br | OH | Cl | CH | CH | CH | CF |
| 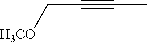 | Br | Cl | CH | CH | CH | CF |
| 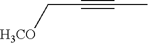 | 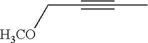 | Cl | CH | CH | CH | CF |
| 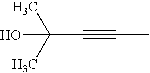 | H | Cl | CH | CH | CH | CF |
| Cl | H | Cl | CH | N | CH | CCl |
| Cl | 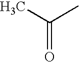 | Cl | CH | CH | CH | CF |
| 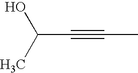 | Br | Cl | CH | CH | CH | CF |
| 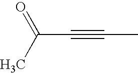 | Br | Cl | CH | CH | CH | CF |
| 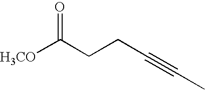 | Br | Cl | CH | CH | CH | CF |
| 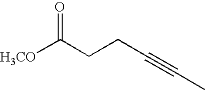 | 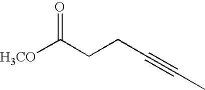 | Cl | CH | CH | CH | CF |
| H | H | Cl | N | CH | CH | N |
| 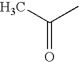 | 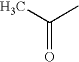 | Cl | CH | CH | CH | CF |
| 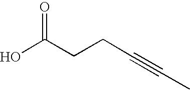 | Br | Cl | CH | CH | CH | CF |
| H | H | Br | N | CH | CH | CH |
| Cl | Br | Cl | CH | CH | CH | CCl |
| Br | 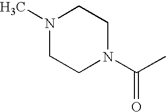 | Cl | CH | CH | CH | CF |
| Cl | H | Br | CH | N | CH | CBr |
| Cl | 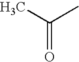 | Cl | CH | CH | CH | CCl |
| H | H | Br | CH | CH | N | CH |
| Cl | H | F | CH | N | CH | CF |
| Br | 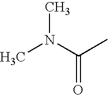 | Cl | CH | CH | CH | CF |
| Br | 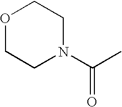 | Cl | CH | CH | CH | CF |
| I | I | I | CH | CH | CH | CF |
| Br | NH₂ | Cl | CH | CH | CH | CF |
| Br | 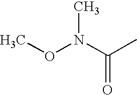 | Cl | CH | CH | CH | CF |
| Br | 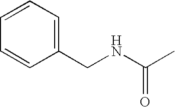 | Cl | CH | CH | CH | CF |
| H | H | Cl | CH | CH | CH | N |
| 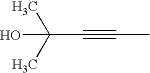 | 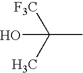 | Cl | CH | CH | CH | CF |
| H | H | Cl | N | CH | CH | CCl |
| 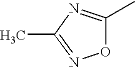 | Br | Cl | CH | CH | CH | CF |
| Br | Cl | Br | CH | CH | CH | CBr |
| Br | 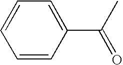 | Cl | CH | CH | CH | CF |
| Cl | 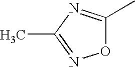 | Cl | CH | CH | CH | CF |
| Cl | 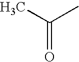 | I | CH | CH | CH | CF |
| Cl | 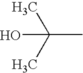 | I | CH | CH | CH | CF |
| Cl | H | Cl | N | CH | CH | CCl |
| Cl | 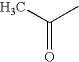 | Br | CH | CH | CH | CBr |
| Cl | 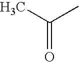 | I | CH | CH | CH | CI |
| Cl | 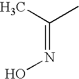 | I | CH | CH | CH | CI |
| Cl | 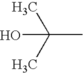 | I | CH | CH | CH | CI |
| I | I | I | CH | CH | CH | CI |
| Br | 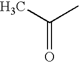 | Cl | CH | CH | CH | CF |
| I | 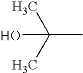 | Cl | CH | CH | CH | CF |
| Cl | 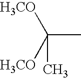 | I | CH | CH | CH | CI |
| Cl | 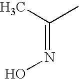 | Br | CH | CH | CH | CBr |
| Cl | 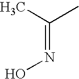 | Cl | CH | CH | CH | CCl |
| Cl | 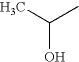 | Br | CH | CH | CH | CBr |
| 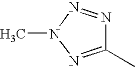 | Cl | Br | CH | CH | CH | CBr |
| Cl | 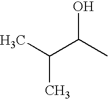 | Cl | CH | CH | CH | CCl |
| Cl | 3-pyridyl | Cl | CH | CH | CH | CCl |
| Cl | 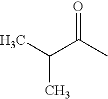 | Cl | CH | CH | CH | CCl |
| 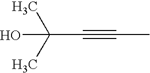 | Cl | Cl | CH | CH | CH | CCl |
| 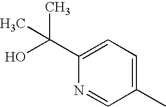 | Cl | Cl | CH | CH | CH | CCl |
| Cl | 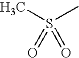 | Br | CH | CH | CH | CBr |
| Br | H | Cl | CH | CH | CH | CCl |
| Br | H | Br | CH | CH | CH | CBr |
| I | H | I | CH | CH | CH | CI |
| Cl | 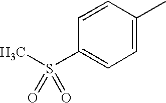 | Br | CH | CH | CH | CBr |
| Cl | 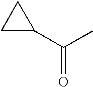 | Br | CH | CH | CH | CBr |
| Cl | 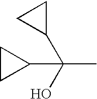 | Br | CH | CH | CH | CBr |
| Cl | 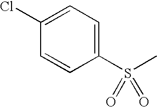 | Br | CH | CH | CH | CBr |
| Cl | F | Br | CH | CH | CH | CBr |
| 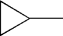 | H | Br | CH | CH | CH | CBr |
| Cl | 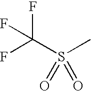 | Br | CH | CH | CH | CBr |
| Cl | 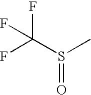 | Br | CH | CH | CH | CBr |
| Cl | 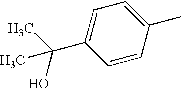 | Br | CH | CH | CH | CBr |
| Cl | 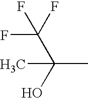 | Br | CH | CH | CH | CBr |
| Br | 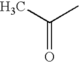 | Br | CH | CH | CH | CBr |
| Br | 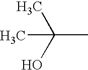 | Br | CH | CH | CH | CBr |
| 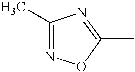 | Br | Br | CH | CH | CH | CBr |
| Cl | Br | Br | CH | N | CH | Br |
| Br | Cl | Br | CH | COH | CH | CBr |
| Br | 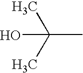 | Br | CH | CH | CH | CBr |
| 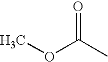 | 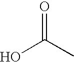 | Br | CH | CH | CH | CBr |

TABLE 2

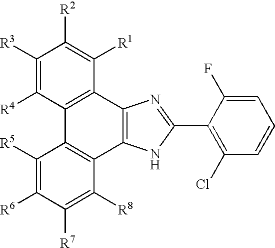

| R¹ | R² | R³ | R⁴ | R⁵ | R⁶ | R⁷ | R⁸ |
|---|---|---|---|---|---|---|---|
| Br | H | H | H | H | H | H | H |
| H | Br | H | H | H | H | H | H |
| H | H | H | Br | H | H | H | H |
| F | H | F | H | H | H | H | H |
| H | Cl | H | H | H | H | H | H |
| H | Cl | H | H | H | H | | H |
| H | Cl | H | H | H | CH₃ | H | H |
| H | Cl | H | H | H | H | H | CH₃ |
| H | Cl | H | H | H | H | 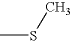 | H |
| H | Cl | H | H | H | H | 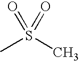 | H |
| H | H | H | H | F | H | F | H |
| H | H | H | H | 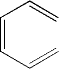 | | H | H |
| H | H | H | H | H | CH₃ | F | H |
| H | H | H | H | H | H | 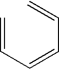 | |
| H | Cl | Cl | H | H | Br | H | H |
| H | H | Cl | Cl | H | Br | H | H |
| H | F | F | F | H | Br | H | H |

or a pharmaceutically acceptable salt of any of the above.

18. A pharmaceutical composition comprising a compound according to claim 1 in combination with a pharmaceutically acceptable carrier.

19. A method for treating a microsomal prostaglandin E synthase-1 mediated disease or condition in a human patient in need of such treatment comprising administering to said patient a compound according to claim 1 in an amount effective to treat the microsomal prostaglandin E synthase-1 mediated disease or condition.

20. The method according to claim 19 wherein the disease or condition is selected from the group consisting of: acute or chronic pain, osteoarthritis, rheumatoid arthritis, bursitis, ankylosing sponylitis and primary dysmenorrhea.

21. The compound according to claim 10, wherein: R³ and R⁶ are independently selected from the group consisting of: hydrogen, fluoro, chloro, bromo, iodo, cyano, methyl, methoxy, ethyl, vinyl, cyclopropyl, propyl, butyl, —CO₂i-Pr, —CO₂CH₃, —SO₂CF₃, 3-pyridyl, acetyl, 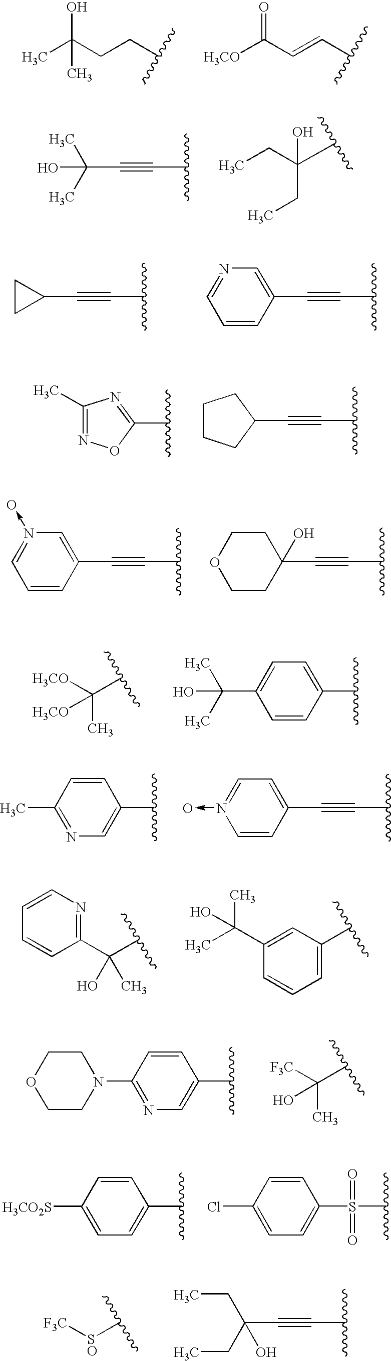 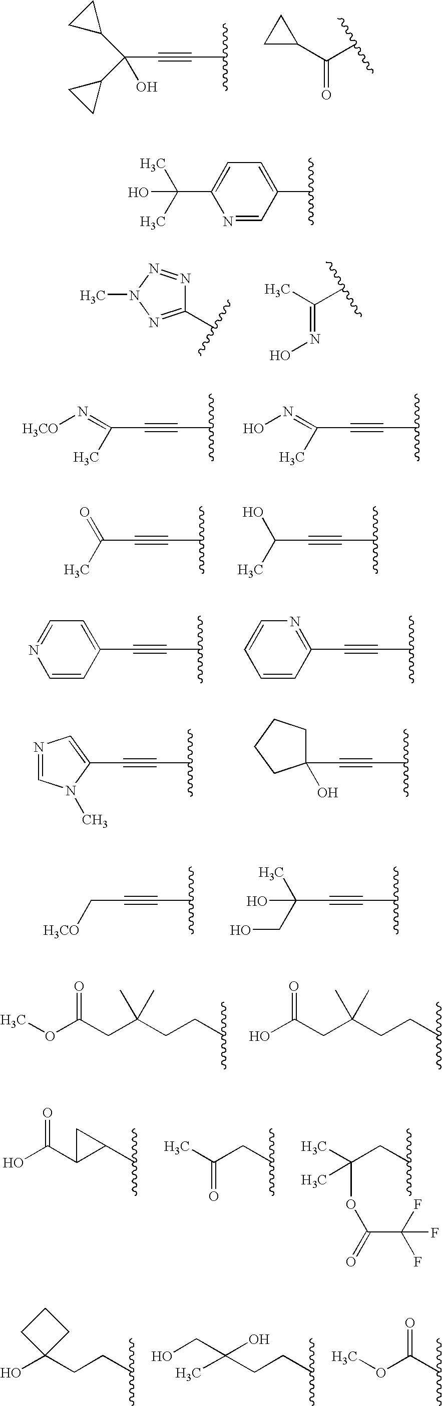 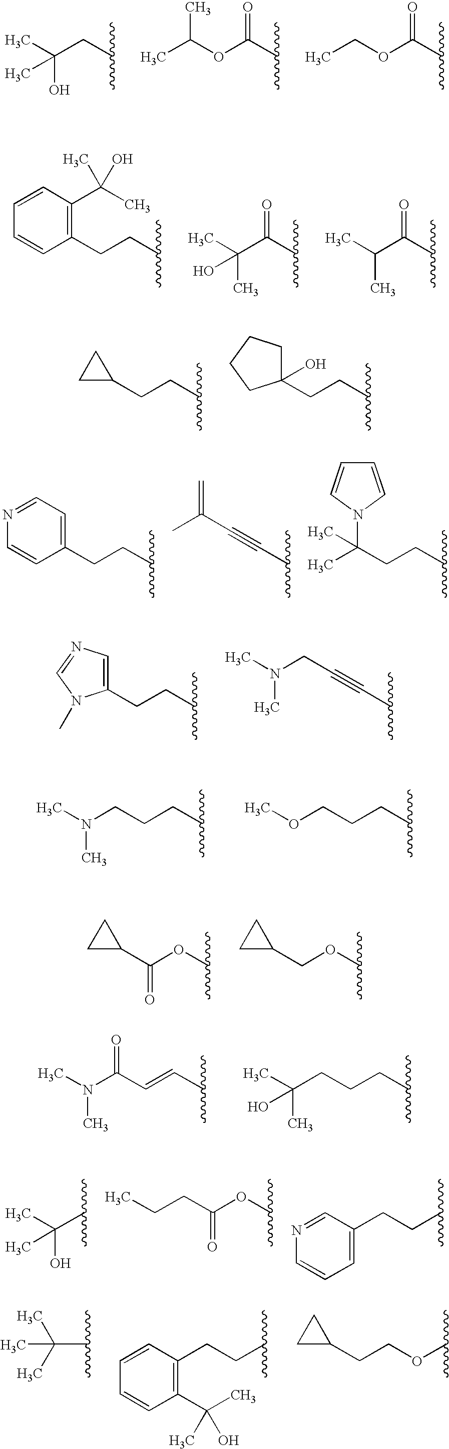 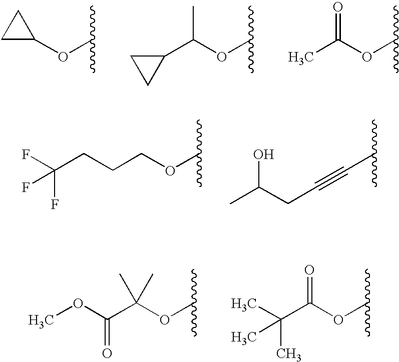
with the proviso that at least one of R³ or R⁶ is other than hydrogen.
